(19) United States
(12) Patent Application Publication
(10) Pub. No.: US 2025/0186106 A1
(43) Pub. Date: Jun. 12, 2025

(54) TREATMENT TOOL

(71) Applicants: Haruhiro INOUE, Kanagawa (JP); OLYMPUS MEDICAL SYSTEMS CORP., Tokyo (JP)

(72) Inventors: Daiki HAGIWARA, Hachioji-shi (JP); Chika MIYAJIMA, Hachioji-shi (JP); Kotaro YAMADA, Hachioji-shi (JP); Haruhiro INOUE, Yokohama-shi (JP)

(21) Appl. No.: 19/057,525

(22) Filed: Feb. 19, 2025

Related U.S. Application Data
(63) Continuation of application No. PCT/JP2023/029159, filed on Aug. 9, 2023.
(60) Provisional application No. 63/373,192, filed on Aug. 22, 2022.

(51) Int. Cl. A61B 18/14; A61B 17/00; A61B 17/29; A61B 18/00

(52) U.S. Cl. CPC A61B 18/1445; A61B 2017/00269; A61B 2017/2943; A61B 2018/00595; A61B 2018/00982

(57) ABSTRACT

A treatment tool includes a sheath, a central axis extending in a width direction orthogonal to a longitudinal direction of the sheath, and forceps having a first forceps piece and a second forceps piece, and in which at least one of the first forceps piece and the second forceps piece is supported to be openable and closable about the central axis, in which a length in the width direction of the first forceps piece and the second forceps piece is equal to or larger than a length in the longitudinal direction of the first forceps piece and the second forceps piece.

The present invention relates to a treatment tool. Priority is claimed on U.S. Provisional Application No. 63/373,192 filed in the United States on Aug. 22, 2022, the content of which is incorporated herein by reference.

### BACKGROUND OF THE INVENTION

### Field of the Invention

### Description of Related Art

Conventionally, in endoscopic therapy, hemostatic forceps (treatment tools) for cauterizing a bleeding treatment target to stop bleeding have been used. The hemostatic forceps described in Patent Document 1 or the like grip a treatment target with openable and closable forceps pieces, and stop bleeding of the treatment target using the forceps pieces through which a high-frequency current is applied.
Patent Document 1: Japanese Patent No. 4668525

### SUMMARY OF THE INVENTION

However, conventional hemostatic forceps (treatment tools) described in Patent Document 1 or the like, due to a longitudinally long cup shape thereof, may cause forceps pieces to push out biological tissues to a distal side, allowing only distal ends of the forceps pieces to grip the biological tissues. Therefore, there has been a likelihood that an operator may not be able to grip biological tissues as intended.

In view of the above circumstances, an objective of the present invention is to provide a treatment tool that makes it easier to grip biological tissues to be treated.

In order to solve the above problems, the present invention proposes the following means.

A treatment tool according to a first aspect of the present invention includes a sheath, a rotation axis extending in a width direction orthogonal to a longitudinal direction of the sheath, and forceps having a first forceps piece and a second forceps piece, and in which at least one of the first forceps piece and the second forceps piece is supported to be openable and closable about the rotation axis, in which a length in the width direction of the first forceps piece and the second forceps piece is equal to or larger than a length in the longitudinal direction of the first forceps piece and the second forceps piece.

According to the treatment tool of the present invention, it is easy to grip biological tissues to be treated.

### BRIEF DESCRIPTION OF THE DRAWINGS

FIG. 1 is an overall view of an endoscopic treatment system according to a first embodiment.

FIG. 2 is an overall view illustrating a treatment tool of the endoscopic treatment system.

FIG. 3 is a perspective view of a distal end part of the treatment tool with forceps in a closed state.

FIG. 4 is a perspective view of the distal end part of the treatment tool with the forceps in a closed state.

FIG. 5 is a perspective view of the distal end part of the treatment tool with the forceps in an open state.

FIG. 6 is a perspective view of the distal end part of the treatment tool with the forceps in an open state.

FIG. 7 is a perspective view of a frame of a support member of the treatment tool.

FIG. 8 is a front view of the treatment tool with the forceps in a closed state.

FIG. 9 is a left side view of the treatment tool with the forceps in a closed state.

FIG. 10 is a left cross-sectional view of the treatment tool with the forceps in a closed state.

FIG. 11 is a plan view of the treatment tool with the forceps in a closed state.

FIG. 12 is a bottom view of the treatment tool with the forceps in a closed state.

FIG. 13 is a right side view of the treatment tool with the forceps in a closed state.

FIG. 14 is a right cross-sectional view of the treatment tool with the forceps in a closed state.

FIG. 15 is a front view of the treatment tool with the forceps in an open state.

FIG. 16 is a left side view of the treatment tool with the forceps in an open state.

FIG. 17 is a left cross-sectional view of the treatment tool with the forceps in an open state.

FIG. 18 is a plan view of the treatment tool with the forceps in a closed state.

FIG. 19 is a bottom view of the treatment tool with the forceps in a closed state.

FIG. 20 is a right side view of the treatment tool with the forceps in an open state.

FIG. 21 is a right cross-sectional view of the treatment tool with the forceps in an open state.

FIG. 22 is a rear view of the distal end part of the treatment tool.

FIG. 23 is a perspective view of a treatment tool according to a second embodiment when forceps are in a closed state.

FIG. 24 is a perspective view of a distal end part of the treatment tool with the forceps in a closed state.

FIG. 25 is a perspective view of the distal end part of the treatment tool with the forceps in an open state.

FIG. 26 is a perspective view of the distal end part of the treatment tool with the forceps in an open state.

FIG. 27 is a left side view of the distal end part of the treatment tool with the forceps in a closed state.

FIG. 28 is a right side view of the distal end part of the treatment tool with the forceps in a closed state.

FIG. 29 is a plan view of the distal end part of the treatment tool with the forceps in a closed state.

FIG. 30 is a bottom view of the distal end part of the treatment tool with the forceps in a closed state.

FIG. 31 is a front view of the distal end part of the treatment tool with the forceps in a closed state.

FIG. 32 is a rear view of the distal end part of the treatment tool.

FIG. 33 is a perspective view of a treatment tool according to a third embodiment in a closed state.

FIG. 34 is a perspective view of a distal end part of the treatment tool with forceps in a closed state.

FIG. 35 is a perspective view of the distal end part of the treatment tool with the forceps in an open state.

FIG. 36 is a perspective view of the distal end part of the treatment tool with the forceps in an open state.

FIG. 37 is a left side view of the distal end part of the treatment tool with the forceps in a closed state.

FIG. 38 is a right side view of the distal end part of the treatment tool with the forceps in a closed state.

FIG. 39 is a plan view of the distal end part of the treatment tool with the forceps in a closed state.

FIG. 40 is a bottom view of the distal end part of the treatment tool with the forceps in a closed state.

FIG. 41 is a front view of the distal end part of the treatment tool with the forceps in a closed state.

FIG. 42 is a rear view of the distal end part of the treatment tool.

FIG. 43 is a perspective view of a distal end part when a treatment tool according to a fourth embodiment is in a closed state.

FIG. 44 is a perspective view of the distal end part of the treatment tool with forceps in a closed state.

FIG. 45 is a perspective view of the distal end part of the treatment tool with the forceps in an open state.

FIG. 46 is a perspective view of the distal end part of the treatment tool with the forceps in an open state.

FIG. 47 is a left side view of the distal end part of the treatment tool with the forceps in a closed state.

FIG. 48 is a right side view of the distal end part of the treatment tool with the forceps in a closed state.

FIG. 49 is a plan view of the distal end part of the treatment tool with the forceps in a closed state.

FIG. 50 is a bottom view of the distal end part of the treatment tool with the forceps in a closed state.

FIG. 51 is a front view of the distal end part of the treatment tool with the forceps in a closed state.

FIG. 52 is a rear view of the distal end part of the treatment tool.

FIG. 53 is a perspective view of a distal end part when a treatment tool according to a fifth embodiment is in a closed state.

FIG. 54 is a perspective view of the distal end part of the treatment tool with forceps in a closed state.

FIG. 55 is a perspective view of the distal end part of the treatment tool with the forceps in an open state.

FIG. 56 is a perspective view of the distal end part of the treatment tool with the forceps in an open state.

FIG. 57 is a left side view of the distal end part of the treatment tool with the forceps in a closed state.

FIG. 58 is a right side view of the treatment tool with the forceps in a closed state.

FIG. 59 is a plan view of the treatment tool with the forceps in a closed state.

FIG. 60 is a bottom view of the treatment tool with the forceps in a closed state.

FIG. 61 is a front view of the treatment tool with the forceps in a closed state.

FIG. 62 is a rear view of the distal end part of the treatment tool.

FIG. 63 is a perspective view of a treatment tool according to a sixth embodiment in a closed state.

FIG. 64 is a perspective view of a distal end part of the treatment tool with forceps in a closed state.

FIG. 65 is a perspective view of the distal end part of the treatment tool with the forceps in an open state.

FIG. 66 is a perspective view of the distal end part of the treatment tool with the forceps in an open state.

FIG. 67 is a left side view of the distal end part of the treatment tool with the forceps in a closed state.

FIG. 68 is a right side view of the distal end part of the treatment tool with the forceps in a closed state.

FIG. 69 is a plan view of the distal end part of the treatment tool with the forceps in a closed state.

FIG. 70 is a bottom view of the distal end part of the treatment tool with the forceps in a closed state.

FIG. 71 is a front view of the distal end part of the treatment tool with the forceps in a closed state.

FIG. 72 is a rear view of the distal end part of the treatment tool.

FIG. 73 is a perspective view of a treatment tool according to a seventh embodiment in a closed state.

FIG. 74 is a perspective view of a distal end part of the treatment tool with forceps in a closed state.

FIG. 75 is a perspective view of the distal end part of the treatment tool with the forceps in an open state.

FIG. 76 is a perspective view of the distal end part of the treatment tool with the forceps in an open state.

FIG. 77 is a left side view of the distal end part of the treatment tool with the forceps in a closed state.

FIG. 78 is a right side view of the distal end part of the treatment tool with the forceps in a closed state.

FIG. 79 is a plan view of the distal end part of the treatment tool with the forceps in a closed state.

FIG. 80 is a bottom view of the distal end part of the treatment tool with the forceps in a closed state.

FIG. 81 is a front view of the distal end part of the treatment tool with the forceps in a closed state.

FIG. 82 is a rear view of the distal end part of the treatment tool with the forceps in a closed state.

FIG. 83 is a perspective view of a treatment tool according to an eighth embodiment in a closed state.

FIG. 84 is a perspective view of a distal end part of the treatment tool with forceps in a closed state.

FIG. 85 is a perspective view of the distal end part of the treatment tool with the forceps in an open state.

FIG. 86 is a perspective view of the distal end part of the treatment tool with the forceps in an open state.

FIG. 87 is a left side view of the distal end part of the treatment tool with the forceps in a closed state.

FIG. 88 is a right side view of the distal end part of the treatment tool with the forceps in a closed state.

FIG. 89 is a plan view of the distal end part of the treatment tool with the forceps in a closed state.

FIG. 90 is a bottom view of the distal end part of the treatment tool with the forceps in a closed state.

FIG. 91 is a front view of the distal end part of the treatment tool with the forceps in a closed state.

FIG. 92 is a rear view of the distal end part of the treatment tool.

FIG. 93 is a perspective view of a treatment tool according to a ninth embodiment when forceps are in a closed state.

FIG. 94 is a perspective view of a distal end part of the treatment tool with the forceps in a closed state.

FIG. 95 is a perspective view of the distal end part of the treatment tool with the forceps in an open state.

FIG. 96 is a perspective view of the distal end part of the treatment tool with the forceps in an open state.

FIG. 97 is a left side view of the distal end part of the treatment tool with the forceps in a closed state.

FIG. 98 is a right side view of the distal end part of the treatment tool with the forceps in a closed state.

FIG. 99 is a plan view of the distal end part of the treatment tool with the forceps in a closed state.

FIG. 100 is a bottom view of the distal end part of the treatment tool with the forceps in a closed state.

FIG. 101 is a front view of the distal end part of the treatment tool with the forceps in a closed state.

FIG. 102 is a rear view of the distal end part of the treatment tool.

FIG. 103 is a perspective view of a treatment tool according to a tenth embodiment when forceps are in a closed state.

FIG. 104 is a perspective view of a distal end part of the treatment tool with the forceps in a closed state.

FIG. 105 is a perspective view of the distal end part of the treatment tool with the forceps in an open state.

FIG. 106 is a perspective view of the distal end part of the treatment tool with the forceps in an open state.

FIG. 107 is a left side view of the distal end part of the treatment tool with the forceps in a closed state.

FIG. 108 is a right side view of the distal end part of the treatment tool with the forceps in a closed state.

FIG. 109 is a plan view of the distal end part of the treatment tool with the forceps in a closed state.

FIG. 110 is a bottom view of the distal end part of the treatment tool with the forceps in a closed state.

FIG. 111 is a front view of the distal end part of the treatment tool with the forceps in a closed state.

FIG. 112 is a rear view of the distal end part of the treatment tool.

FIG. 113 is a view illustrating an example of a use mode of the treatment tool.

### DETAILED DESCRIPTION OF THE INVENTION

### First Embodiment

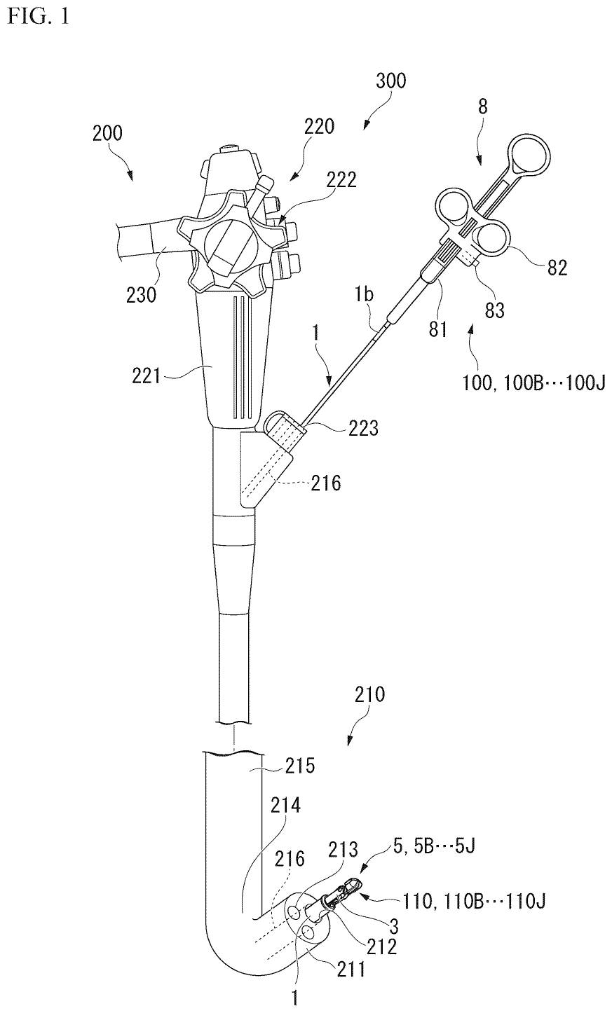

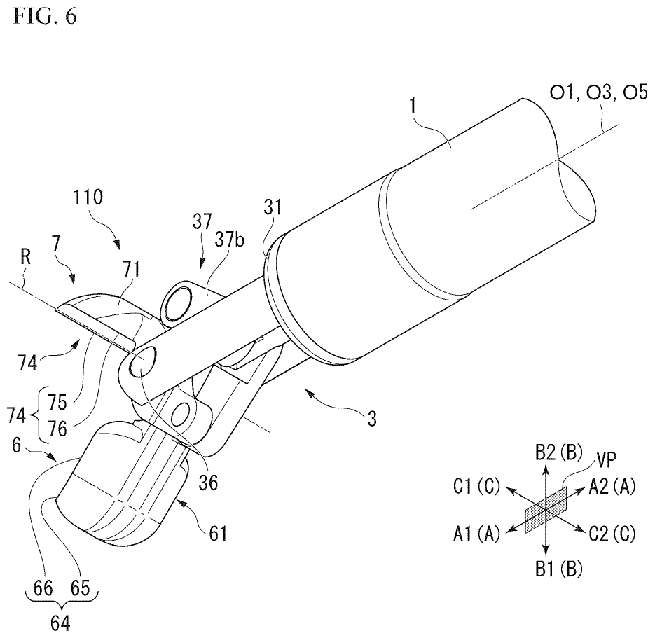

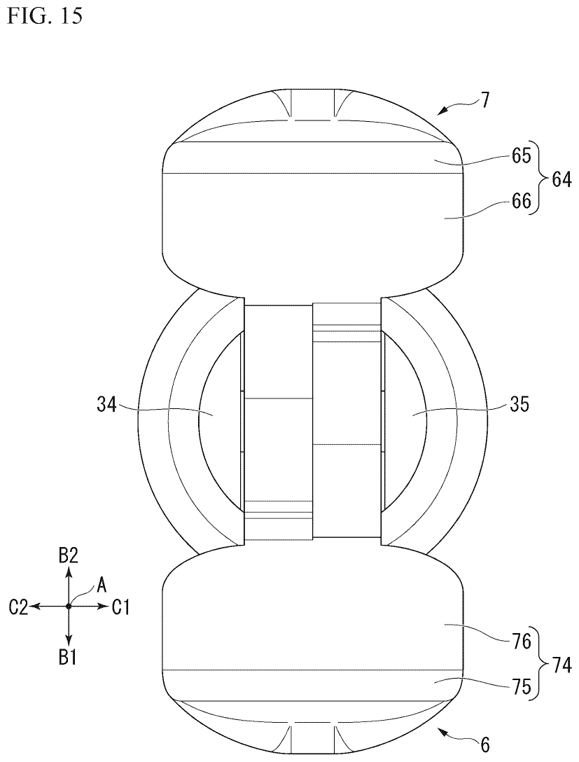

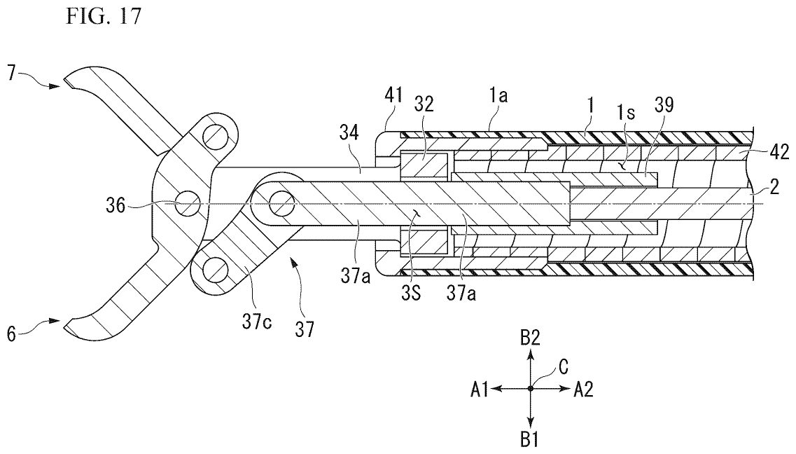

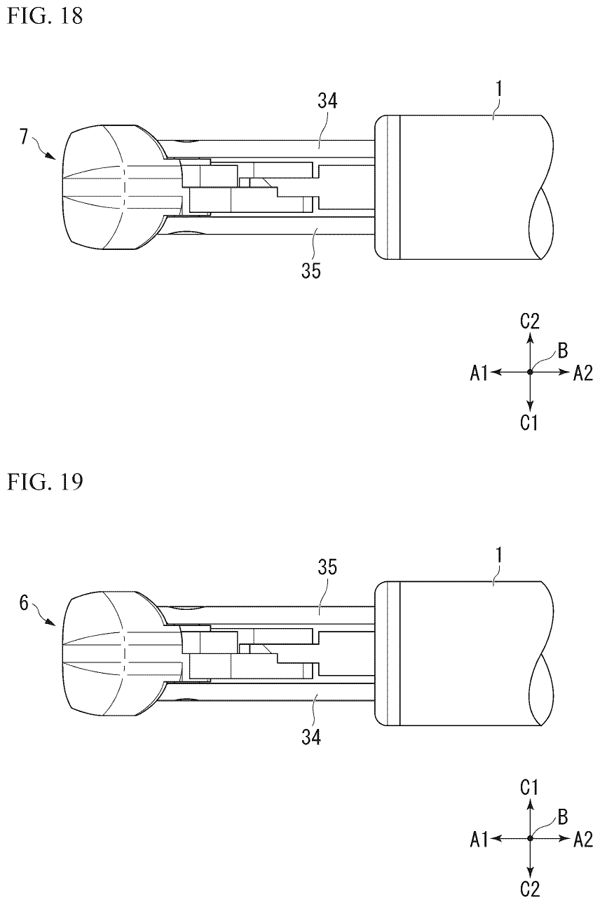

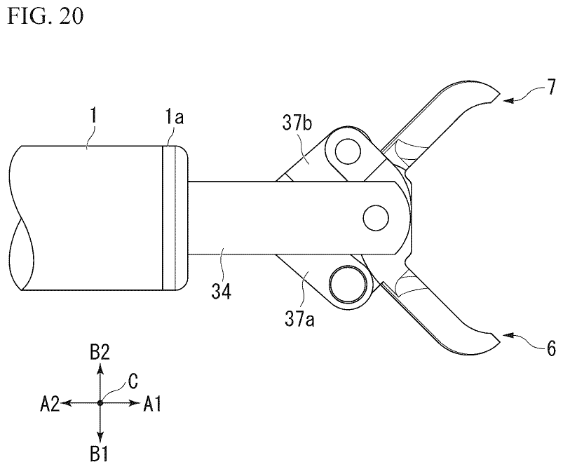

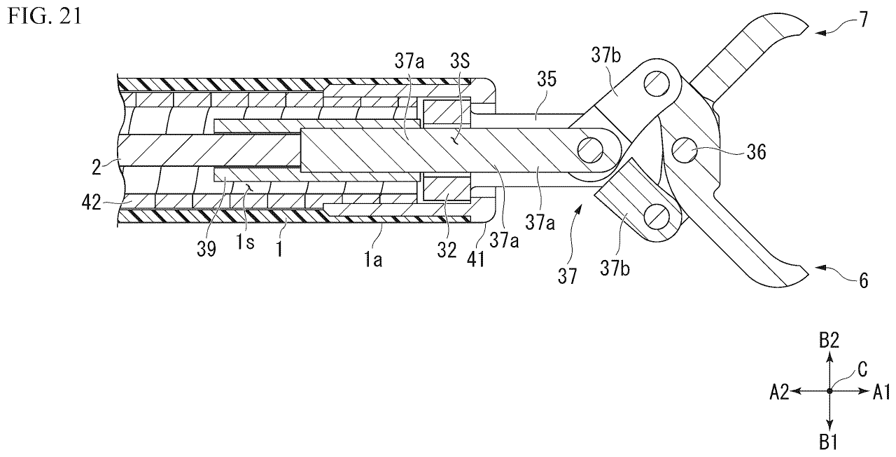

An endoscopic treatment system 300 including an endoscopic treatment tool 100 according to a first embodiment of the present invention will be described with reference to FIGS. 1 to 22. FIG. 1 is an overall view of the endoscopic treatment system 300.

### [Endoscopic Treatment System 300]

As illustrated in FIG. 1, the endoscopic treatment system 300 includes the endoscopic treatment tool 100 and an endoscope 200. The endoscopic treatment tool 100 is used by being inserted into the endoscope 200.

### [Endoscope 200]

The endoscope 200 is a known flexible endoscope and includes an insertion part 210 inserted into a body from a distal end thereof, a manipulation part 220 attached to a proximal end of the insertion part 210, and a universal cord 230 attached to the manipulation part 220.

The insertion part 210 is an elongated member that can be inserted into a lumen. The insertion part 210 has a distal end part 211, a curved part 214, and a flexible part 215. The distal end part 211, the curved part 214, and the flexible part 215 are connected in that order from the distal end side. A channel 216 for inserting the treatment tool 100 is provided inside the insertion part 210. A distal end opening 212 of the channel 216 and an imaging unit 213 are provided in the distal end part 211.

The imaging unit 213 includes an imaging element such as, for example, a CCD or a CMOS and is capable of imaging a portion to be treated. The curved part 214 curves according to a manipulation of the manipulation unit 220 by a user. The flexible part 215 is a tubular portion having flexibility.

The manipulation unit 220 is connected to the flexible part 215. The manipulation unit 220 has a grip 221, an input unit 222, and a forceps port 223. The grip 221 is a member supported by the user. The input unit 222 receives a manipulation input for causing a curving motion of the curved part 214. The forceps port 223 is a proximal end opening of the channel 216.

The universal cord 230 connects the endoscopic treatment tool 100 and an external device. An imaging cable, an optical fiber cable, or the like that outputs an imaging signal captured by the imaging unit 213 to the outside is inserted into the universal cord 230.

### [Endoscopic Treatment Tool 100]

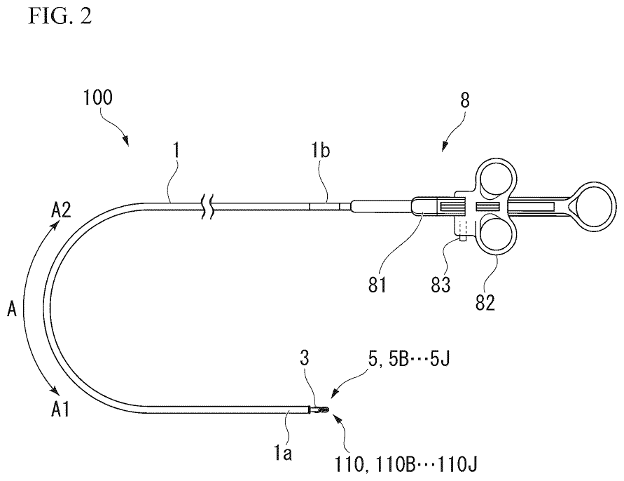

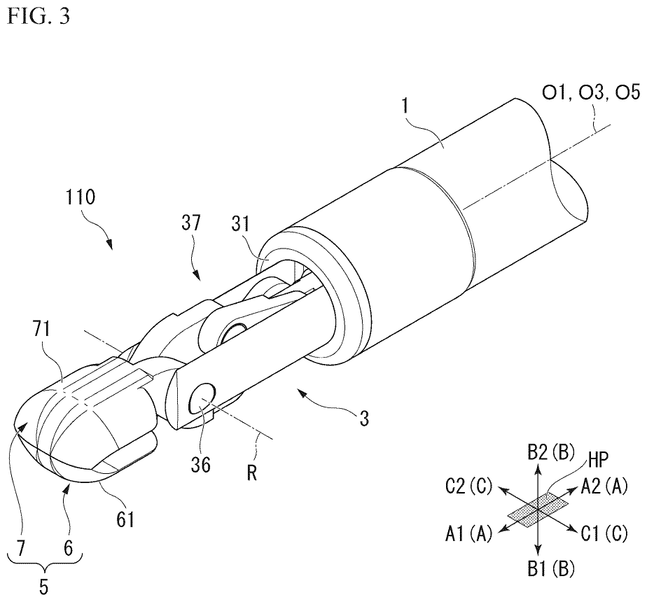

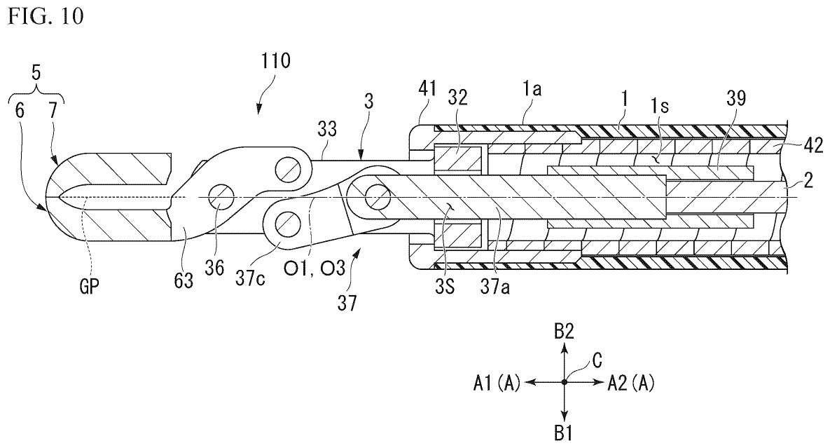

As illustrated in FIG. 2, the endoscopic treatment tool 100 (also referred to as a treatment tool 100) is hemostatic forceps that cauterizes an affected area to stop bleeding. The treatment tool 100 includes a sheath 1, a manipulation wire 2 (see FIG. 10), a support member 3, forceps (jaw) 5, and a manipulation unit 8. In the following description, in a longitudinal direction A of the treatment tool 100, a side inserted into a patient's body is referred to as a “distal end side (distal side) A1,” and a side of the manipulation unit 8 is referred to as a “proximal end side (proximal side) A2”. As illustrated in FIGS. 3 to 6, the support member 3 and the forceps 5 are provided at a distal end part of the treatment tool 100. The forceps 5 are supported by the support member 3. The forceps 5 are supported by the support member 3 to be openable and closable. The support member 3 and the forceps 5 constitute a “treatment part 110” that treats an affected area.

In the following description, a direction in which the forceps 5 open and close is referred to as an “opening/closing direction B” or a “vertical direction B”. Also, a direction perpendicular to the longitudinal direction A and the opening/closing direction B is referred to as a “width direction C” or a “left-right direction C”. Also, a plane that is horizontal to the longitudinal direction A and the width direction C is referred to as a “horizontal plane HP”. A plane that is horizontal to the longitudinal direction A and the opening/closing direction B is referred to as a “vertical plane VP”.

### [Sheath 1]

The sheath 1 is a long member having flexibility and extending from a distal end 1a to a proximal end 1b as illustrated in FIG. 2. The sheath 1 has an outer diameter that can be inserted into the channel 216 of the endoscope 200. As illustrated in FIG. 1, in a state in which the sheath 1 is inserted into the channel 216, the distal end 1a of the sheath 1 can protrude from and retract into the distal end opening 212 of the channel 216. The sheath 1 may have insulating properties.

### [Manipulation Wire 2]

The manipulation wire 2 is a metal wire and is inserted through an internal space 1s of the sheath 1 (see FIG. 10). A distal end of the manipulation wire 2 is connected to a link mechanism 37 of the support member 3, and a proximal end of the manipulation wire 2 is connected to the manipulation unit 8. Further, the link mechanism 37 is not limited to the configuration illustrated in FIG. 10.

### [Support Member 3]

As illustrated in FIG. 2, the support member 3 is attached to the distal end 1a of the sheath 1 and supports the forceps 5. As illustrated in FIGS. 3 to 6, the support member 3 includes a frame 31, a pin (rotating shaft, rotating shaft member) 36, the link mechanism 37, and a coupling part 39. A first forceps piece 6 and a second forceps piece 7 of the forceps 5 are supported by the support member 3 to be openable and closable via the pin 36 attached to the frame 31.

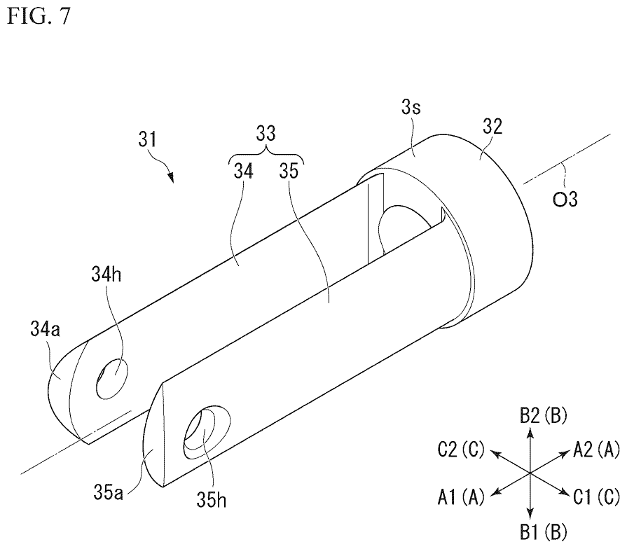

FIG. 7 is a perspective view of the frame 31 of the support member 3.

The frame 31 is made of a metal such as stainless steel and is formed in substantially a U shape. The frame 31 has a support part main body 32 formed in a cylindrical shape, and a pair of frame pieces 33 (a first frame piece 34 and a second frame piece 35).

As illustrated in FIG. 10 or the like, the support part main body 32 is attached to the distal end 1a of the sheath 1 by a clearance fit or the like using a joint part 41 attached to the distal end 1a of the sheath 1. The support part main body 32 is rotatably supported by the joint part 41, that is, the distal end 1a of the sheath 1. A central axis O3 of the support member 3 in the longitudinal direction A substantially coincides with a central axis O1 of the sheath 1 in the longitudinal direction A. An internal space 3s of the support part main body 32 communicates with the internal space Is of the sheath 1 (FIG. 10).

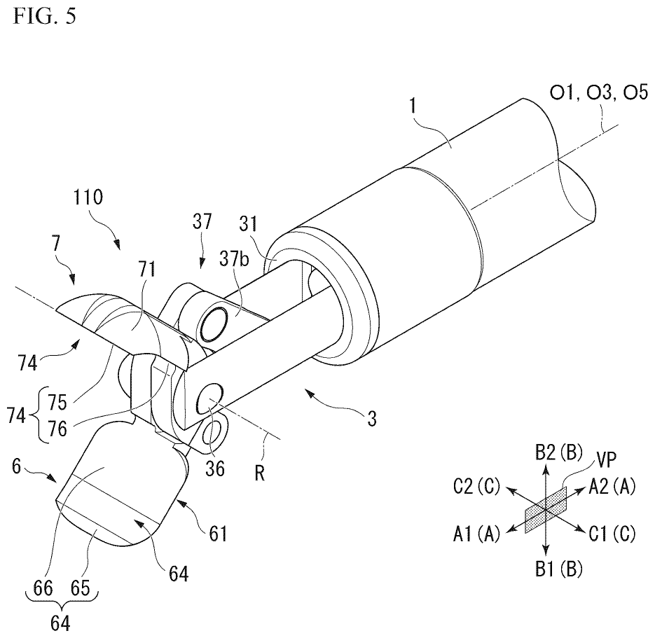

As illustrated in FIG. 5, the pair of frame pieces 33 (the first frame piece 34 and the second frame piece 35) are provided to protrude from the support part main body 32 to the distal end side A1. The first frame piece 34 and the second frame piece 35 are provided evenly on both sides in the width direction C with the central axis O3 therebetween. The first frame piece 34 and the second frame piece 35 have symmetrical shapes with respect to the vertical plane VP that passes through the central axis O3.

The first frame piece 34 is formed in a flat plate shape extending in the longitudinal direction A. A plate thickness direction of the first frame piece 34 substantially coincides with the width direction C. The proximal end side A2 of the first frame piece 34 is continuous with the support part main body 32. A semicircular first distal end part 34a is formed on the distal end side A1 of the first frame piece 34. A first through hole 34h penetrating in the width direction C is formed on the distal end side A1 of the first frame piece 34.

The second frame piece 35 is formed in a flat plate shape extending in the longitudinal direction A. A plate thickness direction of the second frame piece 35 substantially coincides with the width direction C. The proximal end side A2 of the second frame piece 35 is continuous with the support part main body 32. A semicircular second distal end part 35a is formed on the distal end side A1 of the second frame piece 35. A second through hole 35h penetrating in the width direction C is formed on the distal end side A1 of the second frame piece 35.

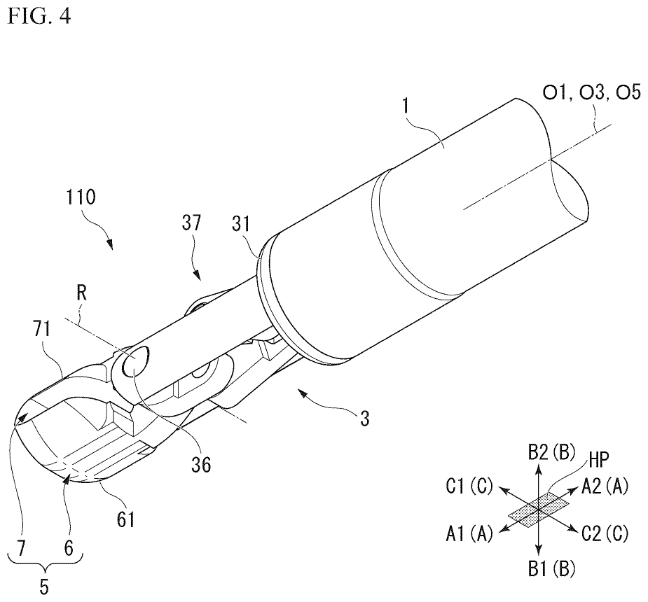

The pin (rotating shaft, rotating shaft member) 36 illustrated in FIGS. 3 and 4 is made of a metal such as stainless steel and has a substantially cylindrical shape. As illustrated in FIG. 5, the pin 36 is attached to the frame 31 by being inserted into and engaged with the first through hole 34h and the second through hole 35h.

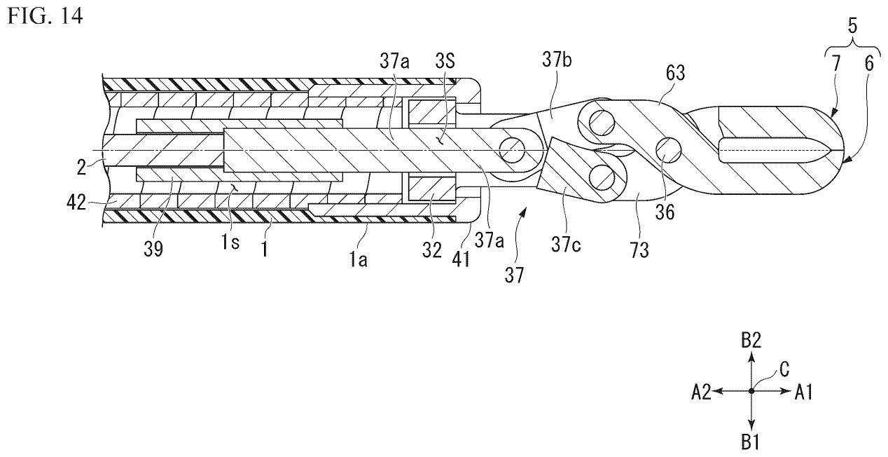

The link mechanism 37 is a link mechanism that converts an advancing and retracting motion of the manipulation wire 2 in the longitudinal direction A illustrated in FIGS. 10 and 14 into an opening and closing motion of the forceps 5 as illustrated in FIGS. 3 to 6. As illustrated in FIGS. 10, 14, and the like, the link mechanism 37 has a rod 37a, a first link 37b, and a second link 37c.

The rod 37a is formed in a cylindrical shape and is inserted into the internal space 3s of the support part main body 32. The rod 37a can advance and retract in the longitudinal direction A. A distal end of the manipulation wire 2 is attached to the proximal end side A2 of the rod 37a.

The first link 37b is a plate-shaped member that couples the rod 37a and the first forceps piece 6 of the forceps 5. The first link 37b is coupled to the rod 37a to be rotatable with respect to the rod 37a along the vertical plane VP. Also, the first link 37b is coupled to the first forceps piece 6 to be rotatable with respect to the first forceps piece 6 along the vertical plane VP.

The second link 37c is a plate-shaped member that couples the rod 37a and the second forceps piece 7 of the forceps 5. The second link 37c is coupled to the rod 37a to be rotatable with respect to the rod 37a along the vertical plane VP. Also, the second link 37c is coupled to the second forceps piece 7 to be rotatable with respect to the second forceps piece 7 along the vertical plane VP.

Further, the manipulation wire 2 may be directly coupled to the proximal end side of the coupling part 39, or may be coupled via a coupling material (not illustrated).

### [Forceps (Jaw) 5]

The forceps (jaw) 5 is a member for gripping biological tissues. As illustrated in FIGS. 3 and 4, the forceps 5 are supported by the support member 3 to be openable and closable toward the distal end side A1. The forceps 5 are formed of a metal material such as stainless steel, and include the first forceps piece 6 and the second forceps piece 7.

The first forceps piece 6 is supported by the pin 36 to be rotatable about a central axis R of the pin 36, which extends in a width direction orthogonal to the longitudinal direction of the sheath 1, as a rotation center. The first forceps piece 6 is manipulated by the first link 37b coupled to the proximal end side A2 of the first forceps piece 6.

The second forceps piece 7 is supported by the pin 36 to be rotatable about the central axis R of the pin 36 as a rotation center. The second forceps piece 7 is manipulated by the second link 37c coupled to the proximal end side A2 of the second forceps piece 7.

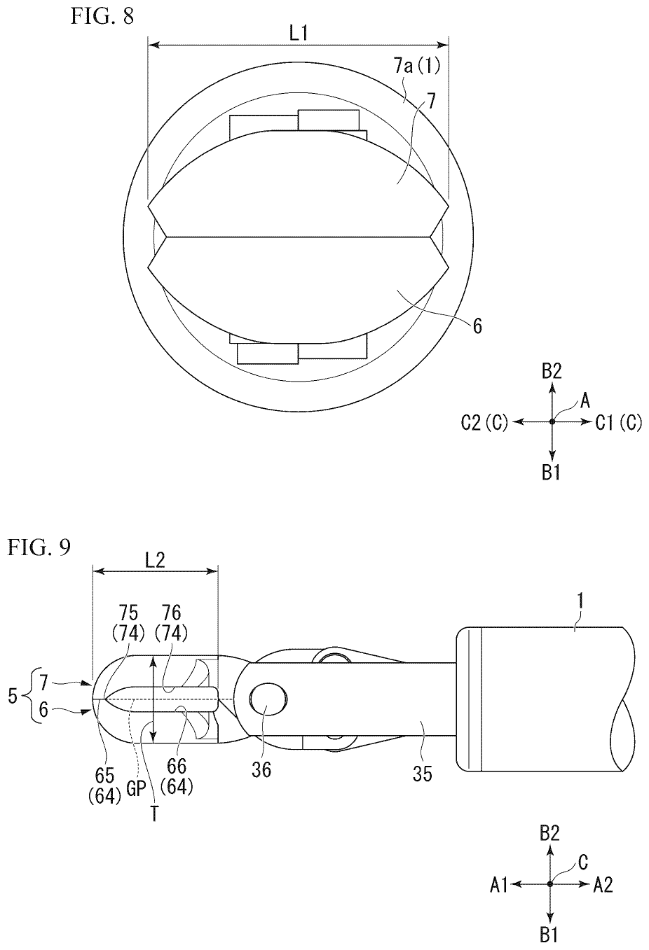

The first forceps piece 6 and the second forceps piece 7 are disposed to be symmetrical with respect to a central axis O5 of the forceps 5 in the longitudinal direction A (see FIGS. 8 and 9). A central axis O5 of the forceps 5 in the longitudinal direction A substantially coincides with the central axis O1 of the sheath 1 in the longitudinal direction A.

Further, one of the first forceps piece 6 and the second forceps piece 7 may be fixed to the support member 3, and only the other may be rotatably supported by the support member 3. Also, the forceps 5 may be directly manipulated by the manipulation wire instead of the link mechanism 37.

In the following description, a direction in which the first forceps piece 6 opens is referred to as a “lower side B1” in the opening/closing direction B, and a direction in which the second forceps piece 7 opens is referred to as an “upper side B2” in the opening/closing direction B. Also, a direction facing right when viewed from the distal end side A1 to the proximal end side A2 is referred to as a “right side C1” in the width direction C, and a direction facing left is referred to as a “left side C2” in the width direction C.

### [First Forceps Piece 6]

As illustrated in FIGS. 4, 9, and 10, the first forceps piece 6 has a first gripping part 61 provided on the distal end side A1, and a first plate 63.

The first gripping part 61 is formed in a semi-ellipsoidal shape (see FIG. 8) on the lower side B1, and has a first gripping surface 64 on the upper side B2 for gripping biological tissues.

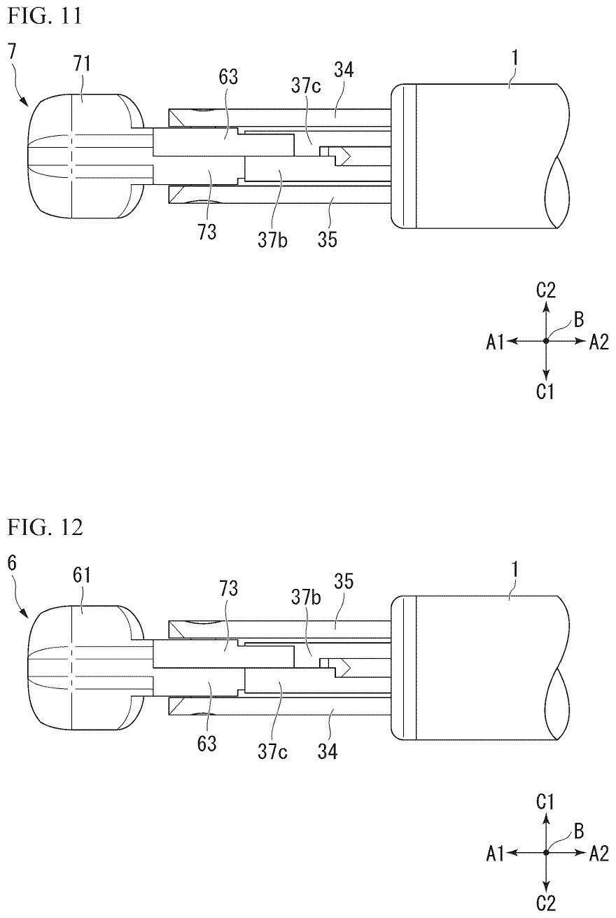

The first plate 63 is provided on the proximal end side A2 of the first gripping part 61 and is formed in substantially a plate shape as illustrated in FIG. 10. A plate thickness direction of the first plate 63 substantially coincides with the width direction C. As illustrated in FIGS. 11 and 12, the first plate 63 is disposed adjacent to a second plate 73 of the second forceps piece 7 on the right side C1.

As illustrated in FIGS. 4, 9, and 10, the first plate 63 is supported by the pin 36 to be rotatable about the central axis R of the pin 36 as a rotation center. Also, a proximal end part of the first plate 63 is rotatably coupled to a distal end part of the first link 37b.

### [Second Forceps Piece 7]

As illustrated in FIGS. 5, 13, and 14, the second forceps piece 7 has a second gripping part 71 provided on the distal end side A1, and the second plate 73.

The second gripping part 71 is formed in a semi-ellipsoidal shape (see FIG. 8) on the upper side B2, and has a second gripping surface 74 on the lower side B1 for gripping biological tissues.

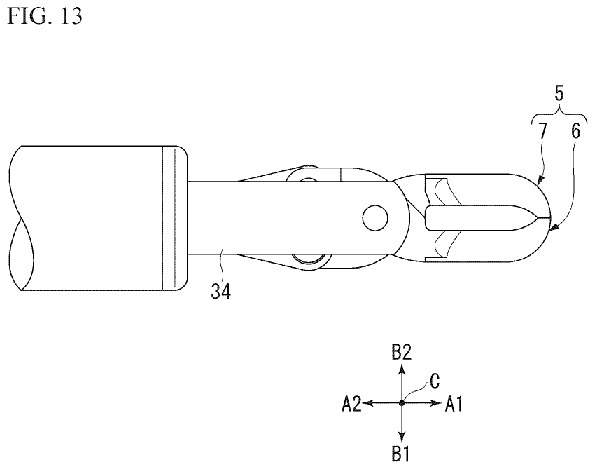

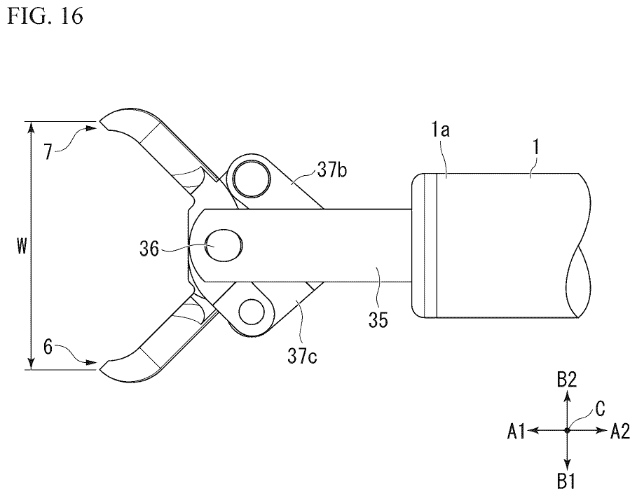

The second plate 73 is provided on the proximal end side A2 of the second gripping part 71, and is formed in substantially a plate shape as illustrated in FIG. 16. A plate thickness direction of the second plate 73 substantially coincides with the width direction C. As illustrated in FIGS. 13 and 14, the second plate 73 is disposed adjacent to the first plate 63 of the first forceps piece 6 on the left side C2.

As illustrated in FIG. 14, the second plate 73 is supported by the pin 36 to be rotatable about the central axis R of the pin 36 as a rotation center. Also, a proximal end part of the second plate 73 is rotatably coupled to a distal end part of the second link 37c.

### [First Gripping Surface 64 and Second Gripping Surface 74]

As illustrated in FIGS. 9 and 10, the first gripping surface 64 and the second gripping surface 74 are surfaces facing each other when the forceps 5 are in a closed state, and at least a part of the surfaces come into contact with each other on the horizontal plane HP (hereinafter also referred to as a “gripping plane GP”) passing through the central axis O5 of the forceps 5. The first gripping surface 64 and the second gripping surface 74 are formed in a shape symmetrical with respect to the gripping plane GP.

The first gripping surface 64 is a surface with an unevenness, and as illustrated in FIG. 9, has a first contact protruding part 65 that comes into contact with the second gripping surface 74 of the second forceps piece 7 when the forceps 5 are in a closed state, and a first accommodating recessed part 66 that does not come into contact with the second gripping surface 74 when the forceps 5 are in a closed state.

As illustrated in FIG. 9, the first contact protruding part 65 is provided at a distal end of the first gripping surface 64. The first contact protruding part 65 is formed in a substantially planar shape extending in the width direction C along the gripping plane GP. The first contact protruding part 65 comes into contact with a second contact protruding part 75 of the second gripping surface 74 that faces the first contact protruding part 65 in the opening/closing direction B when the forceps 5 are in a closed state.

As illustrated in FIG. 9, the first accommodating recessed part 66 is a recessed part recessed from the gripping plane GP to the lower side B1 when the forceps 5 are in a closed state, and is provided on a proximal end side with respect to the first contact protruding part 65. The first accommodating recessed part 66 is formed in a substantially planar shape on the lower side B1 of the gripping plane GP. The first accommodating recessed part 66 curves toward the upper side B2 at a distal end side thereof and is smoothly connected to the proximal end side of the first contact protruding part 65.

The second gripping surface 74 is a surface with an unevenness, and as illustrated in FIG. 9, has the second contact protruding part 75 that comes into contact with the first gripping surface 64 of the first forceps piece 6 when the forceps 5 are in a closed state, and a second accommodating recessed part 76 that does not come into contact with the first gripping surface 64 when the forceps 5 are in a closed state.

As illustrated in FIG. 9, the second contact protruding part 75 is provided at a distal end of the second gripping surface 74. The second contact protruding part 75 is formed in a substantially planar shape extending in the width direction C along the gripping plane GP. The second contact protruding part 75 comes into contact with the first contact protruding part 65 of the first gripping surface 64 that faces the second contact protruding part 75 in the opening/closing direction B when the forceps 5 are in a closed state.

As illustrated in FIG. 9, the second accommodating recessed part 76 is a recessed part recessed from the gripping plane GP to the upper side B2 when the forceps 5 are in a closed state, and is provided on a proximal end side with respect to the second contact protruding part 75. The second accommodating recessed part 76 is formed in a substantially planar shape.

As illustrated in FIG. 9, when the forceps 5 are in a closed state, only the first contact protruding part 65 and the second contact protruding part 75 come into contact with each other. Therefore, a distance between the first gripping surface 64 of the first forceps piece 6 and the second gripping surface 74 of the second forceps piece 7 is larger on the proximal end side than on the distal end side in the longitudinal direction A.

A maximum length L1 (FIG. 8) in the width direction C of the forceps 5 (the first forceps piece 6 and the second forceps piece 7) in the present embodiment is within a range of 1.0 times or more to 1.6 times or less a length L2 (FIG. 9) of the forceps 5 in the longitudinal direction A, with 1.1 times being particularly preferable. Also, the maximum length L1 in the width direction C of the forceps 5 (the first forceps piece 6 and the second forceps piece 7) is equal to or smaller than a maximum outer diameter of the sheath 1.

An opening width W (FIG. 16) when the forceps 5 of the present embodiment are maximally opened is within a range of 1.7 times or more to 2.8 times or less the length L2 (FIG. 9) of the forceps 5 in the longitudinal direction A, with 2.0 times being particularly preferable.

A length T in the thickness direction B orthogonal to the longitudinal direction A when the forceps 5 of the present embodiment are closed is in a range of 0.2 times or more to 0.6 times or less the length L2 of the forceps 5 in the longitudinal direction A, with 0.3 times being particularly preferable.

Also, the length L1 (FIG. 8) of the forceps 5 (the first forceps piece 6 and the second forceps piece 7) in the width direction C is smaller than an outer diameter of the sheath 1.

### [Manipulation Unit 8]

As illustrated in FIG. 2, the manipulation unit 8 is provided on the proximal end side A2 of the sheath 1. The manipulation unit 8 has a manipulation unit main body 81, a slider 82, and a power supply connector 83.

A distal end part of the manipulation unit main body 81 is connected to the proximal end 1b of the sheath 1. The manipulation unit main body 81 has an internal space through which the manipulation wire 2 can be inserted. The manipulation wire 2 passes through the internal space of the sheath 1 and the internal space of the manipulation unit main body 81 and extends to the slider 82.

The slider 82 is attached to the manipulation unit main body 81 to be movable in the longitudinal direction A. A proximal end part of the manipulation wire 2 is connected to the slider 82. When an operator advances or retracts the slider 82 relative to the manipulation unit main body 81, the manipulation wire 2 advances or retracts.

Also, when the operator rotates the manipulation unit 8, the slider 82 rotates the manipulation wire 2, and the forceps 5 rotate accordingly.

The power supply connector 83 is fixed to the slider 82.

The power supply connector 83 can be connected to a high-frequency power supply device (not illustrated), and is electrically and physically connected to the proximal end part of the manipulation wire 2. The power supply connector 83 is capable of supplying a high-frequency current, which is supplied from the high-frequency power supply device, to the forceps 5 via the manipulation wire 2 and the support member 3.

### [Method of Use of the Endoscopic Treatment System 300]

Next, a procedure using the endoscopic treatment system 300 (a method of use of the endoscopic treatment system 300) of the present embodiment illustrated in FIG. 1 will be described. Specifically, incision/dissection treatments, and a hemostasis treatment for a lesion in an endoscopic therapy such as endoscopic submucosal dissection (ESD) will be described.

Incision/dissection treatments are often accompanied by bleeding. In the event of bleeding, the operator performs a hemostasis treatment. The hemostatic treatment is a treatment for stopping bleeding by cauterizing an ulcerated area after a lesion has been dissected or a bleeding site that occurs during incision or dissection treatment.

The operator grips biological tissues including a bleeding site with the first gripping surface 64 and the second gripping surface 74 of the forceps 5. The forceps 5 are provided at a distal end thereof with the first contact protruding part 65 and the second contact protruding part 75 which come into contact with each other when the forceps 5 are closed. Therefore, when the operator closes the forceps 5 to clamp the biological tissues between the first gripping surface 64 and the second gripping surface 74, the biological tissues are less likely to shift to the distal end side A1 with respect to the forceps 5. As a result, the operator can suitably grip the bleeding site between the first accommodating recessed part 66 and the second accommodating recessed part 76 of the forceps 5.

The operator applies a high-frequency current to the manipulation wire 2 in a state in which the living tissues are clamped by the forceps 5. The high-frequency current is applied to each of the first gripping surface 64 and the second gripping surface 74 that are in contact with the biological tissues, thereby cauterizing the bleeding site. The operator continues the above-described operation (treatment) as necessary, and finally excises the lesion to end the ESD procedure.

According to the endoscopic treatment tool 100 of the present embodiment, when the biological tissues to be treated are gripped by the first gripping surface 64 and the second gripping surface 74 of the forceps 5, the biological tissues are less likely to be pushed out to the distal end side (distal side) A1, making it easier to grip the biological tissues to be treated.

Also, the forceps 5 (the first forceps piece 6 and the second forceps piece 7) are formed in a horizontally long cup shape, thereby making it possible to grip a wider range of biological tissues than in conventional cases.

While the first embodiment of the present invention has been described in detail with reference to the drawings, the specific configurations are not limited to the embodiment and may include design changes or the like within a range not departing from the gist of the present invention. Also, the components illustrated in the embodiment and modified examples described above can be configured by appropriately combining them.

Configurations of other embodiments of the treatment part 110 used in the endoscopic treatment tool 100 of the present invention will be described below.

### Second Embodiment

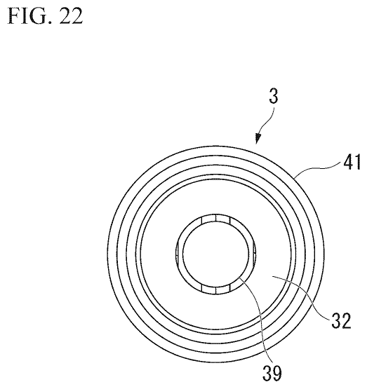

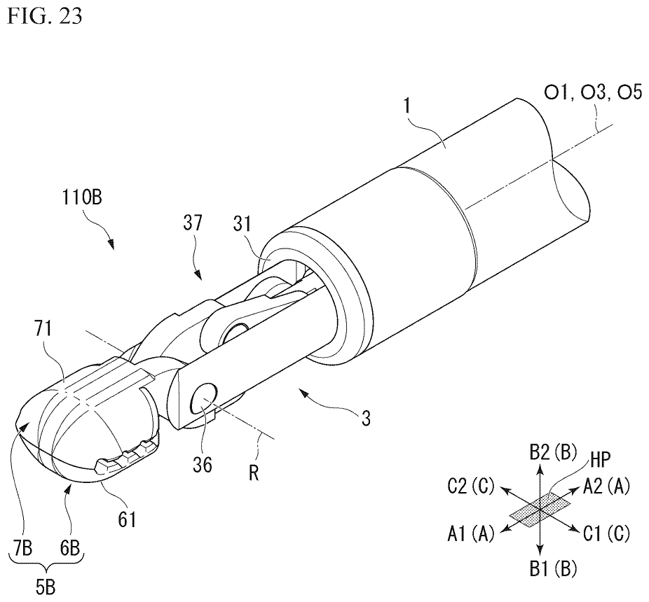

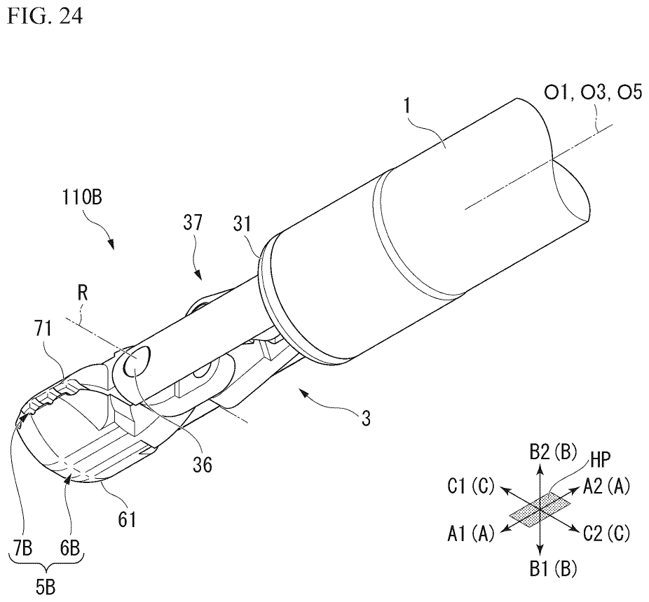

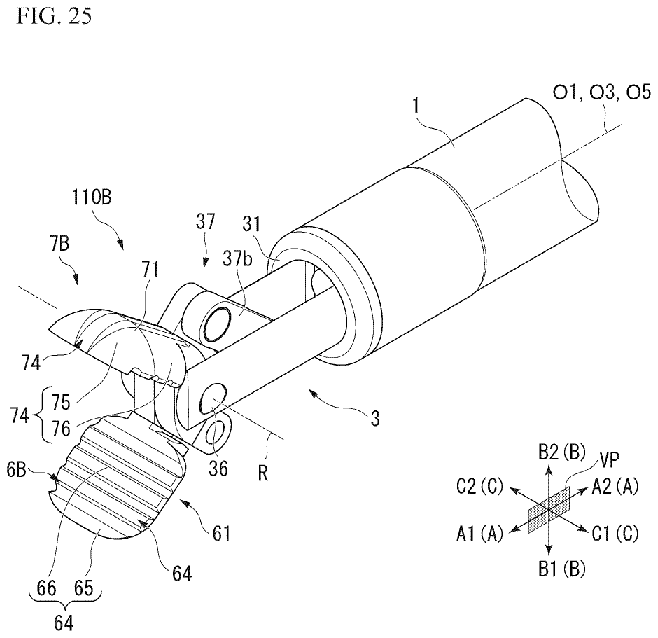

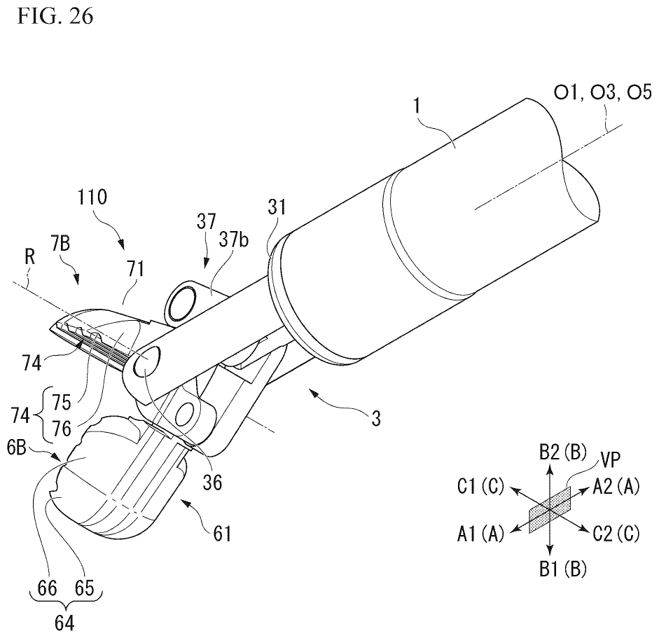

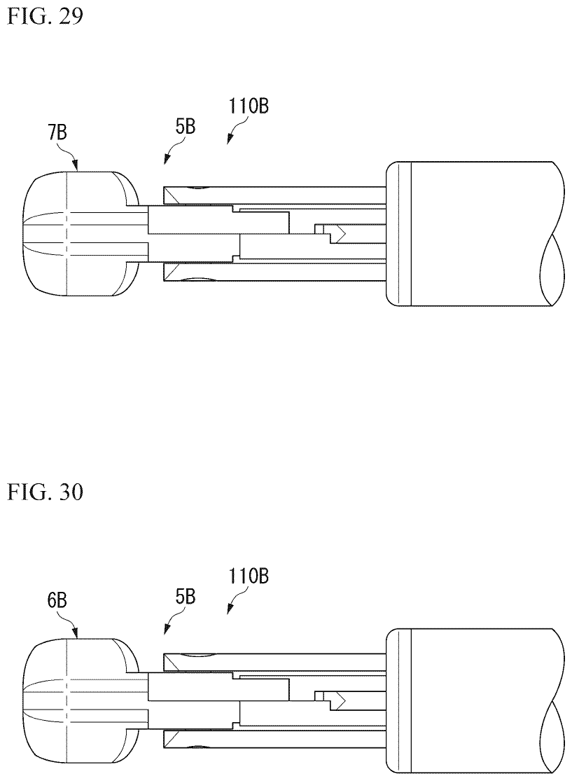

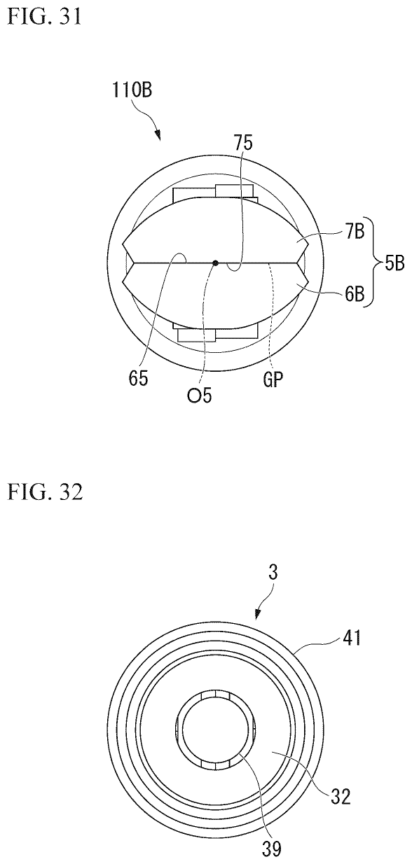

A treatment part 110B according to a second embodiment of the present invention will be described with reference to FIGS. 23 to 32. In the following description, components that are common to those already described will be denoted by the same reference signs and duplicate description will be omitted.

### [Endoscopic Treatment Tool 100B]

An endoscopic treatment tool 100B (also simply referred to as a treatment tool 100B) of the second embodiment illustrated in FIG. 1, together with an endoscope 200, is used as an endoscopic treatment system similarly to the endoscopic treatment tool 100 of the first embodiment. The treatment tool 100B includes a sheath 1, a manipulation wire 2, a support member 3, forceps 5B, and a manipulation unit 8 similarly to the embodiment described above.

### [Treatment Part 110B]

As illustrated in FIGS. 23 to 31, the treatment part 110B positioned at a distal end of the treatment tool 100B of the second embodiment includes the forceps 5B.

The forceps 5B of the present embodiment include a first forceps piece 6B and a second forceps piece 7B. When the forceps 5B are placed in a closed state, biological tissues can be gripped between a first gripping part 61B of the first forceps piece 6B and a second gripping part 71B of the second forceps piece 7B.

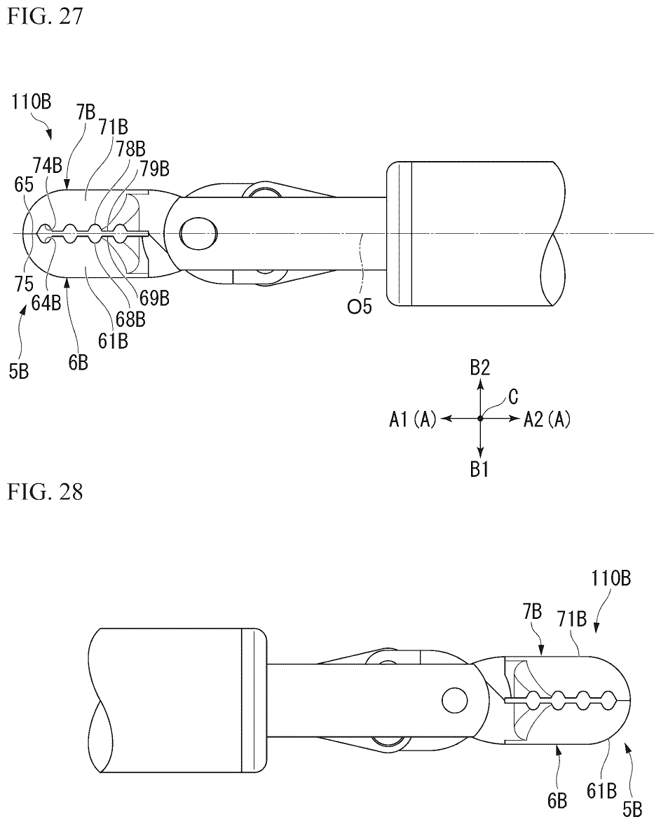

The first gripping part 61B has a first gripping surface 64B that can face the second forceps piece 7B. The first gripping surface 64B is a surface with an unevenness in an opening/closing direction B, and as illustrated in FIG. 27, has a plurality of recessed parts 68B and a plurality of tooth parts (first tooth parts) 69B on a proximal end side of a first contact protruding part 65. The recessed parts 68B and the tooth parts 69B are alternately provided in a longitudinal direction A of the first gripping part 61B.

The second gripping part 71B has a second gripping surface 74B that can face the first forceps piece 6B. The second gripping surface 74B is a surface with an unevenness in the opening/closing direction B, and as illustrated in FIG. 27, has a plurality of recessed parts 78B and a plurality of tooth parts (second tooth parts) 79B on a proximal end side of a second contact protruding part 75. The recessed parts 78B and the tooth parts 79B are alternately provided in the longitudinal direction A of the second gripping part 71B.

The first gripping part 61B and the second gripping part 71B of the present embodiment have shapes symmetrical to each other with a central axis O5 of the forceps 5B therebetween, and the recessed parts 68B and 78B and the tooth parts 69B and 79B, respectively, have shapes that are equal to each other.

The recessed parts 68B and 78B have a groove shape extending throughout a width direction C of the forceps pieces 6B and 7B, and open to outer circumferential surfaces (side surfaces on both sides) of the first forceps piece 6B and the second forceps piece 7B. The recessed parts 68B and 78B have a semicircular shape when viewed from the width direction C of the forceps 5B, but a shape of the recessed parts 68B and 78B is not limited thereto.

The tooth parts 69B and 79B have a protruding shape extending throughout the width direction C of the gripping parts 61B and 71B. Both side surfaces of the tooth parts 69B and 79B in the width direction C are inclined inward as they approach tooth distal ends in the opening/closing direction B. Top edges of the plurality of tooth parts 69B and the plurality of tooth parts 79B form a planar shape parallel to the central axis O5 when the forceps 5B are in a closed state. When the forceps 5B are in a closed state, distances from the top edges of the plurality of tooth parts 69B and the plurality of tooth part 79B to the central axis O5 are all equal regardless of positions in the longitudinal direction A.

The recessed parts 68B and the recessed parts 78B coincide with each other in positions in the longitudinal direction A of the forceps 5B. The tooth parts 69B and the tooth parts 79B are also coincide with each other in positions in the longitudinal direction A of the forceps 5B. Therefore, when the forceps 5 are in a closed state, each recessed part 68 of the first gripping part 61B faces each recessed part 78 of the second gripping part 71B in the opening/closing direction B, and each tooth part 69B of the first gripping part 61B faces each tooth part 79B of the second gripping part 71B in the opening/closing direction B.

When the forceps 5B are in a closed state, contact surfaces of the first contact protruding part 65 and the second contact protruding part 75 coincide with the central axis O5 of the forceps 5B. On the other hand, each tooth part 69B of the first gripping part 61B and each tooth part 79B of the second gripping part 71B do not come into contact with each other even when the forceps 5B are in a completely closed state, and a slight gap occurs between them. In other words, the tooth parts 69B and 79B are positioned further in the vertical direction B away from the central axis O5 of the forceps 5B (the first forceps piece 6B and the second forceps piece 7B) in the longitudinal direction A than a gripping plane GP at which the first gripping surface 64B and the second gripping surface 74B come into contact. The gap described above is smaller than a gap formed between the recessed parts 68B and 78B.

According to the forceps 5B of the present embodiment, when biological tissues to be treated are gripped by the first gripping surface 64B and the second gripping surface 74B of the forceps 5B, the unevenness formed by the recessed parts 68B and 78B and the tooth parts 69B and 79B makes it more difficult for the biological tissues to be pushed out to a distal end side (distal side) A1, making it easier to grip the biological tissues to be treated more stably.

### Third Embodiment

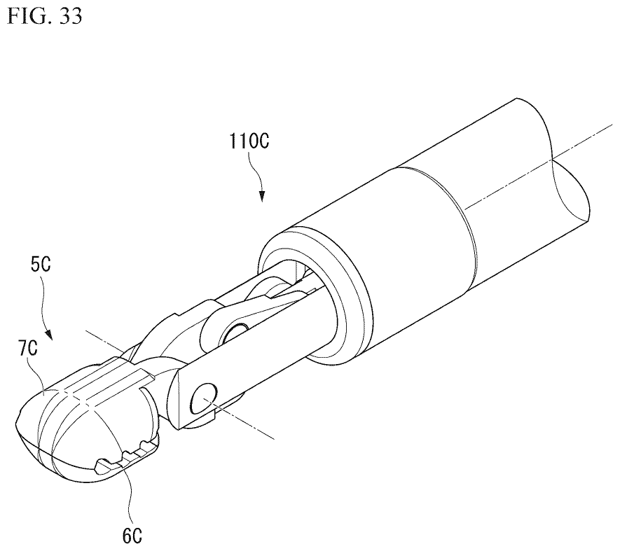

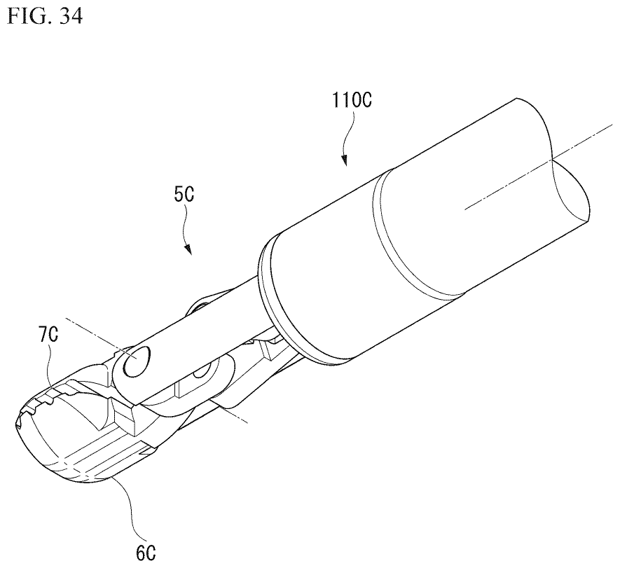

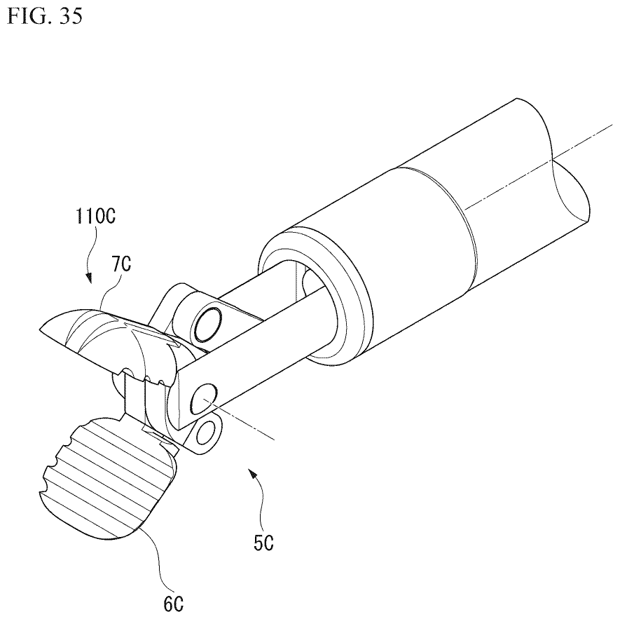

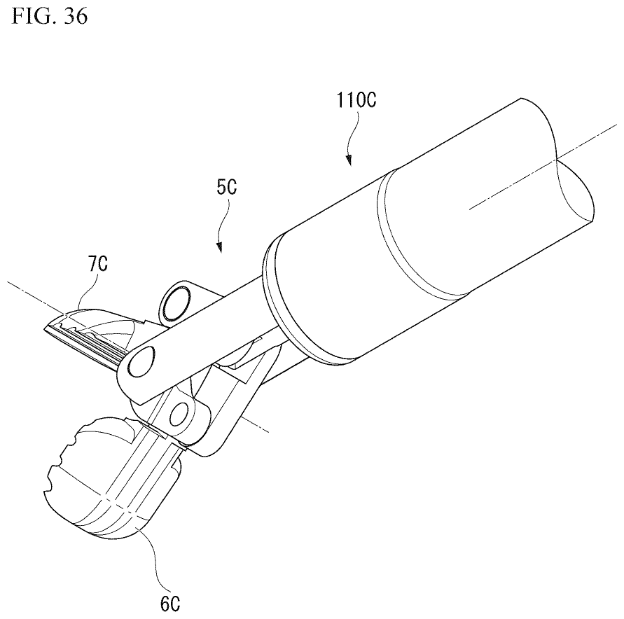

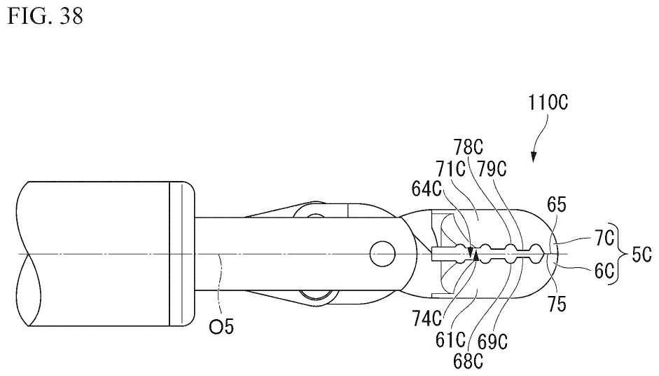

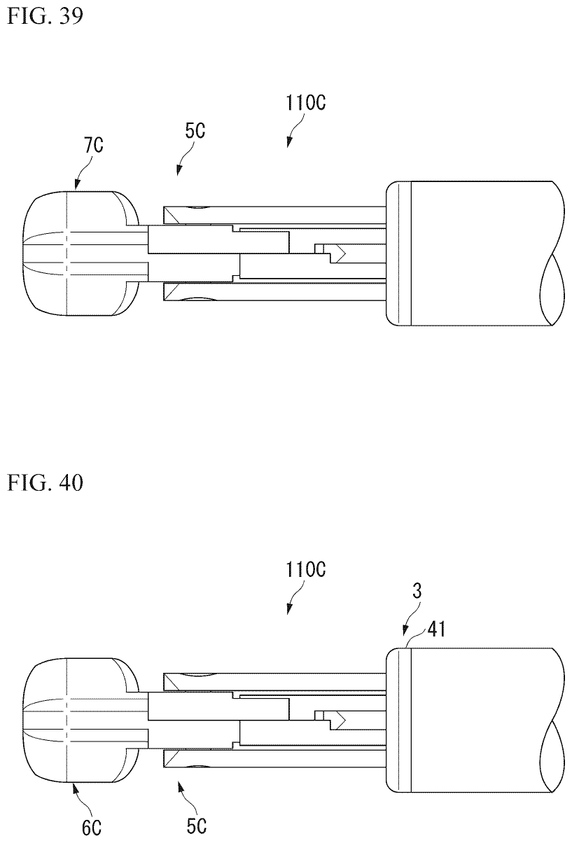

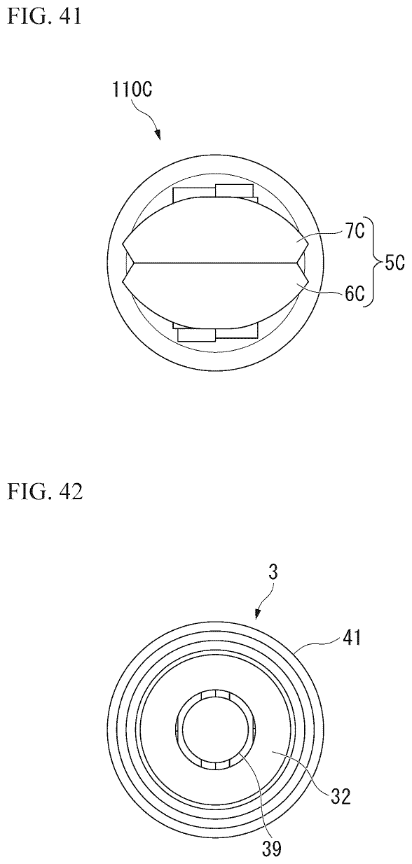

A treatment part 110C according to a third embodiment of the present invention will be described with reference to FIGS. 33 to 42. In the following description, components that are common to those already described will be denoted by the same reference signs and duplicate description will be omitted.

### [Endoscopic Treatment Tool 100C]

An endoscopic treatment tool 100C (also simply referred to as a treatment tool 100C) of the third embodiment illustrated in FIG. 1, together with an endoscope 200, is used as an endoscopic treatment system similarly to the endoscopic treatment tool 100 of the first embodiment. The treatment tool 100C includes a sheath 1, a manipulation wire 2, a support member 3, forceps 5C, and a manipulation unit 8.

### [Treatment Part 110C]

The treatment part 110C positioned at a distal end of the treatment tool 100C of the third embodiment includes the forceps 5C.

The forceps 5C of the present embodiment include a first forceps piece 6C and a second forceps piece 7C. When the forceps 5C are placed in a closed state, biological tissues can be gripped between a first gripping part 61C of the first forceps piece 6C and a second gripping part 71C of the second forceps piece 7C.

The first gripping part 61C has a first gripping surface 64C that can face the second forceps piece 7C. The first gripping surface 64C is a surface with an unevenness in an opening/closing direction B, and has a plurality of recessed parts 68C and a plurality of tooth parts 69C (first tooth parts) on a proximal end side of a first contact protruding part 65 that comes into contact with a second gripping surface 74C. The recessed parts 68C and the tooth parts 69C are alternately provided in a longitudinal direction A of the first gripping part 61C.

The second gripping part 71C has the second gripping surface 74C that can face the first forceps piece 6C. The second gripping surface 74C is a surface with an unevenness in the opening/closing direction B, and has a plurality of recessed parts 78C and a plurality of tooth parts (second tooth parts) 79C on a proximal end side of a second contact protruding part 75. The recessed parts 78C and tooth parts 79C are provided alternately in the longitudinal direction A of the second gripping part 71C.

The recessed parts 68C and 78C of the present embodiment have a trapezoidal shape that widens toward a central axis O5 in a side view from a width direction C. Height positions of bottom surfaces of the plurality of recessed parts 68C aligned in the longitudinal direction A are equal to each other, and distances thereof from the central axis O5 are also equal. Similarly, height positions of bottom surfaces of the plurality of recessed parts 78C aligned in the longitudinal direction A are equal to each other, and distances thereof from the central axis O5 are also equal. Also, the bottom surfaces of the plurality of recessed parts 68C and 78C are all parallel to the central axis O5.

In the present embodiment, the tooth parts 69C and 79C have a trapezoidal shape that widens in a direction away from the central axis O5 in a side view from the width direction C. Height positions of top edges of the plurality of tooth parts 69C and 79C aligned in the longitudinal direction A are different from each other. That is, as the tooth parts 69C and 79C are positioned further on the proximal end side, heights of their top edges decrease and they are positioned farther away from the central axis O5.

The first gripping part 61C and the second gripping part 71C of the present embodiment have shapes symmetrical to each other with the central axis O5 of the forceps 5C therebetween, but heights of the top edges of the plurality of tooth parts 69C (tooth parts 79C) aligned in the longitudinal direction A decrease toward the proximal end side.

Specifically, the tooth parts 69C (the tooth parts 79C) positioned further on the proximal end side have the top edges farther away from the central axis O5 of the forceps 5C when the forceps 5C are in a closed state. That is, a longest distance between the tooth part 69C of the first forceps piece 6C and the tooth part 79C of the second forceps piece 7C which face each other when the forceps 5C are in a closed state is a distance between the tooth parts 69C and 79C positioned on a most proximal end side, and the distance between the tooth parts 69C and 79C increases as the tooth parts 69C and 79C are positioned further on the proximal end side.

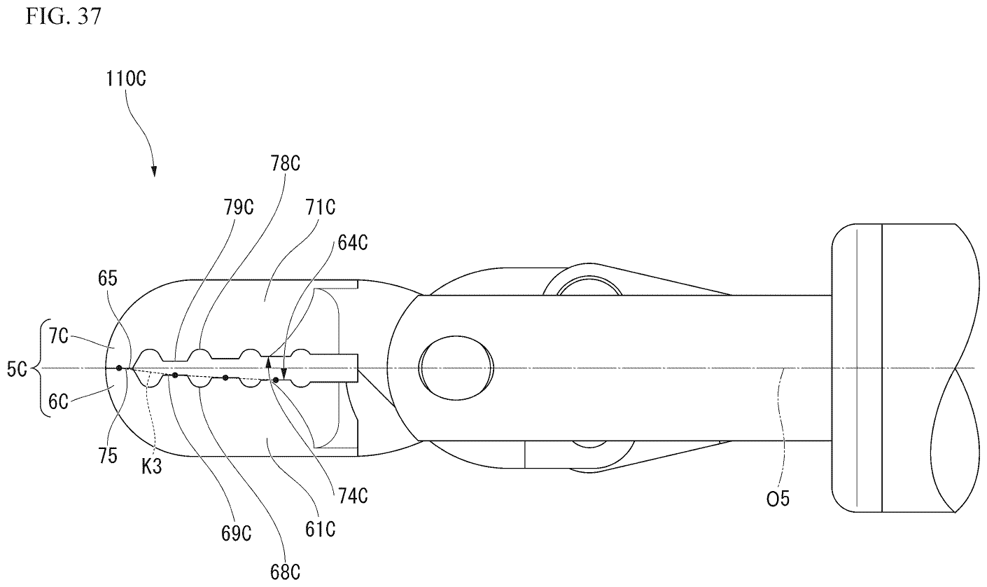

As illustrated in FIG. 37, a virtual line K3 connecting midpoints of the top edges of the plurality of tooth parts 69C aligned in the longitudinal direction A is inclined at a predetermined angle with respect to the central axis O5 and forms a straight line that becomes lower (farther away from the central axis O5) on the proximal end side than on the distal end side of the forceps 5C. Similarly, a virtual line (not illustrated) connecting midpoints of the top edges of the plurality of tooth parts 79C aligned in the longitudinal direction A is also inclined at the same angle as the above-described virtual line K3 with respect to the central axis O5.

Also, the tooth parts 69C and 79C positioned at the most proximal end side have a larger width of the top edge in the longitudinal direction A than the other tooth parts 69C and 79C.

According to the forceps 5C of the present embodiment, when the forceps 5C are in a closed state, since the gap between the tooth parts 69C and 79C becomes wider toward the proximal end side, a space for accommodating a target tissue on a rear end side (proximal side) of the forceps 5C is larger than that in the second embodiment, making it possible to grip the target tissue better without letting it escape.

### Fourth Embodiment

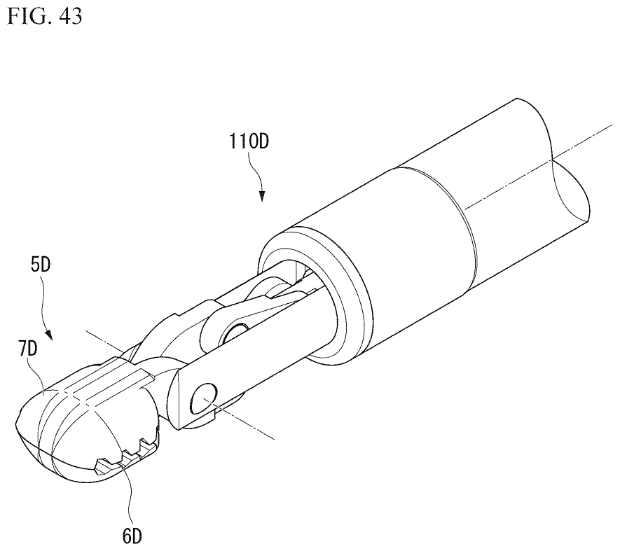

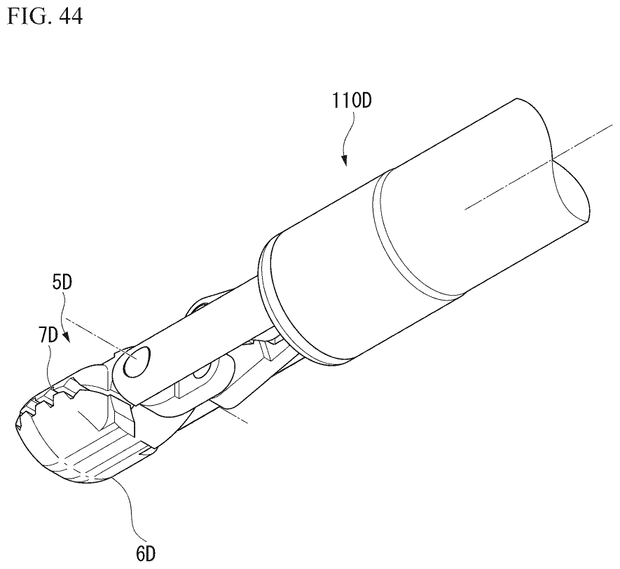

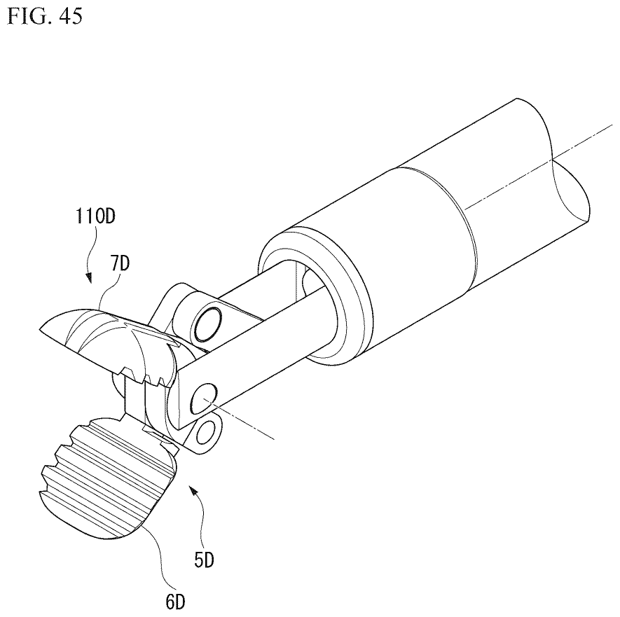

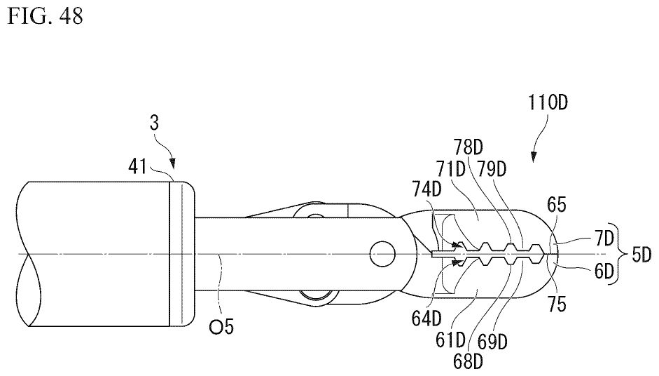

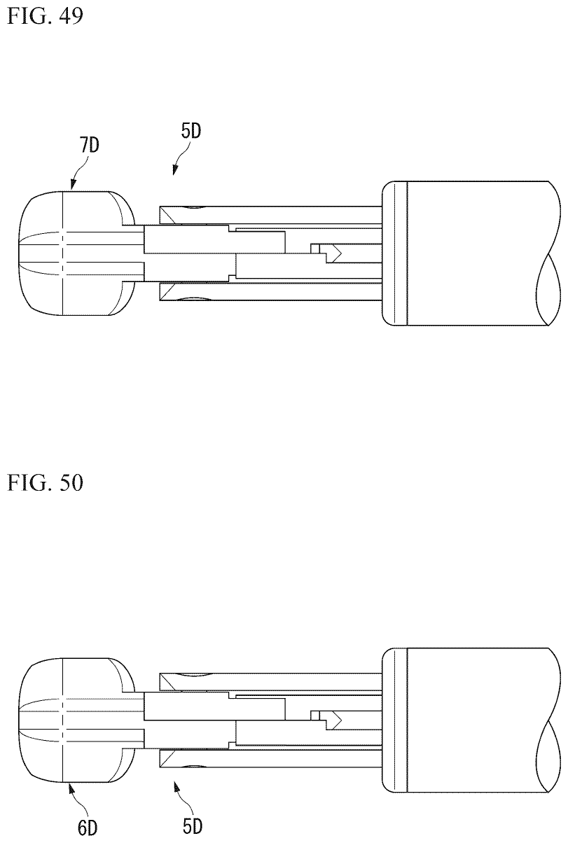

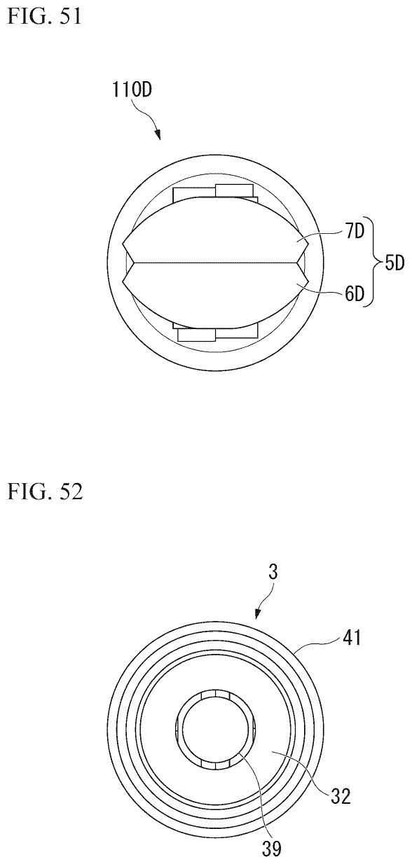

A treatment part 110D according to a fourth embodiment of the present invention will be described with reference to FIGS. 43 to 52. In the following description, components that are common to those already described will be denoted by the same reference signs and duplicate description will be omitted.

### [Endoscopic Treatment Tool 100D]

An endoscopic treatment tool 100D (also simply referred to as a treatment tool 100D) of the fourth embodiment illustrated in FIG. 1, together with an endoscope 200, is used as an endoscopic treatment system similarly to the endoscopic treatment tool 100 of the first embodiment. The treatment tool 100D includes a sheath 1, a manipulation wire 2, a support member 3, forceps 5D, and a manipulation unit 8.

### [Treatment Part 110D]

The treatment part 110D positioned at a distal end of the treatment tool 100D of the fourth embodiment includes the forceps 5D.

The forceps 5D of the present embodiment include a first forceps piece 6D and a second forceps piece 7D. When the forceps 5D are placed in a closed state, biological tissues can be gripped between a first gripping part 61D of the first forceps piece 6D and a second gripping part 71D of the second forceps piece 7D.

The first gripping part 61D has a first gripping surface 64D that can face the second forceps piece 7D. The first gripping surface 64D is a surface with an unevenness in an opening/closing direction, and has a plurality of recessed parts 68D and a plurality of tooth parts (first tooth parts) 69D on a proximal end side of a first contact protruding part 65 that comes into contact with a second gripping surface 74D. The recessed parts 68D and the tooth parts 69D are provided alternately in a longitudinal direction A of the first gripping part 61D.

The second gripping part 71C has the second gripping surface 74D that can face the first forceps piece 6D. The second gripping surface 74D is a surface with an unevenness in the opening/closing direction, and has a plurality of recessed parts 78D and a plurality of tooth parts (second tooth parts) 79D on a proximal end side of a second contact protruding part 75. The recessed parts 78D and the tooth parts 79D are provided alternately in the longitudinal direction A of the second gripping part 71D.

In the present embodiment, in a closed state, the plurality of tooth parts 69D of the first gripping part 61D and the plurality of tooth parts 79D of the second gripping part 71D do not come into contact with each other, and gaps are formed between them. The above-described gaps between the tooth parts 69D and the tooth parts 79D facing each other in a closed state are all equal regardless of positions in the longitudinal direction A. That is, distances from top edges of the plurality of tooth parts 69D and the plurality of tooth parts 79D to a central axis O5 are equal, and the top edges are parallel to the central axis O5.

On the other hand, bottom surfaces of the recessed parts 68D and 78D become lower (farther away from the central axis O5) toward the proximal end side. That is, the recessed parts 68D and 78D positioned further on the proximal end side have a shape with a deeper groove, and the recessed parts 68D and 78D positioned on the most proximal end side are the deepest and are also farthest from the central axis O5.

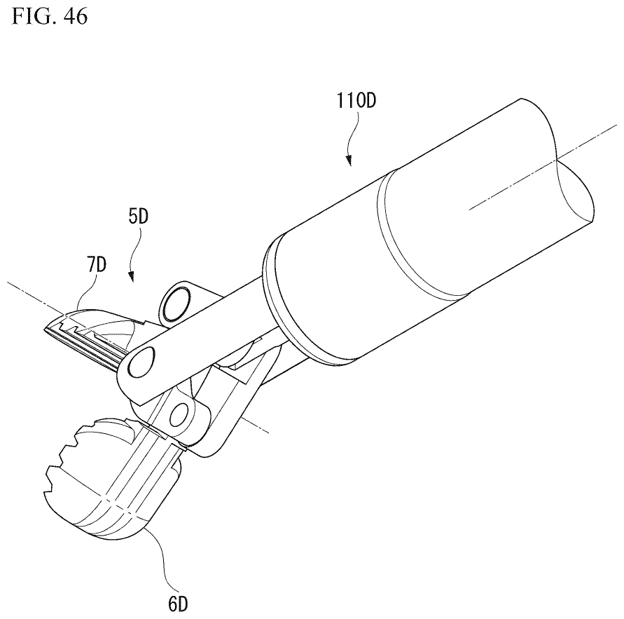

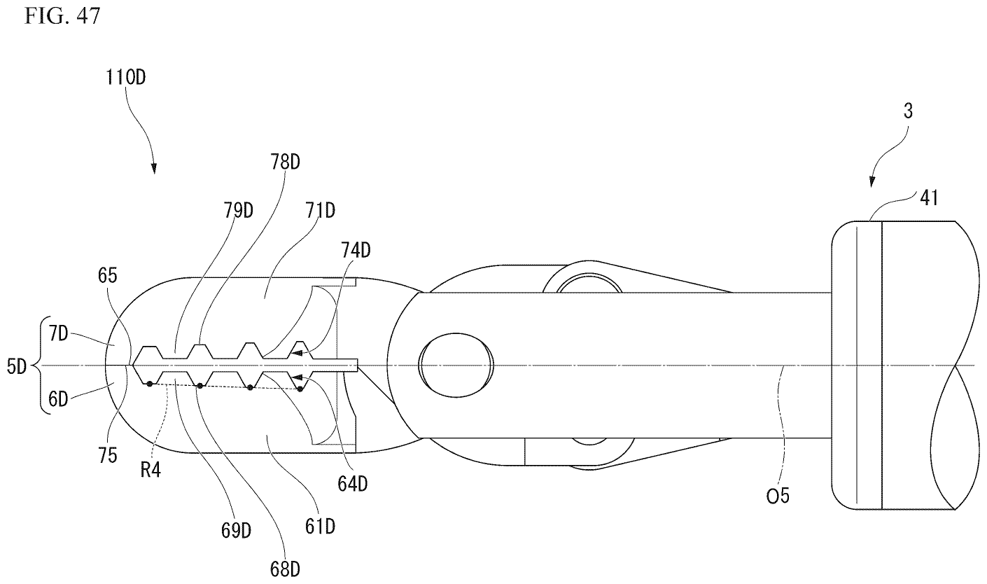

As illustrated in FIG. 47, a virtual line R4 connecting midpoints of the bottom surfaces of the plurality of recessed parts 68D aligned in the longitudinal direction A is inclined at a predetermined angle with respect to the central axis O5 and forms a straight line that is lower on the proximal end side than on the distal end side of the forceps 5D. Similarly, a virtual line (not illustrated) connecting midpoints of the top edges of the plurality of tooth parts 79D aligned in the longitudinal direction A is also inclined at the same angle as the above-described virtual line R4 with respect to the central axis O5.

Also, widths of the top edges of the plurality of tooth parts 69D and 79D in the longitudinal direction A are all equal.

According to the forceps 5D of the present embodiment, when the forceps 5D are in a closed state, since the recessed parts 68D and 78D that become deeper toward the proximal end side face each other, a space for accommodating a target tissue on a rear end side (proximal side) of the forceps 5D is larger than that in the second embodiment, making it possible to grip the target tissue better without letting it escape.

### Fifth Embodiment

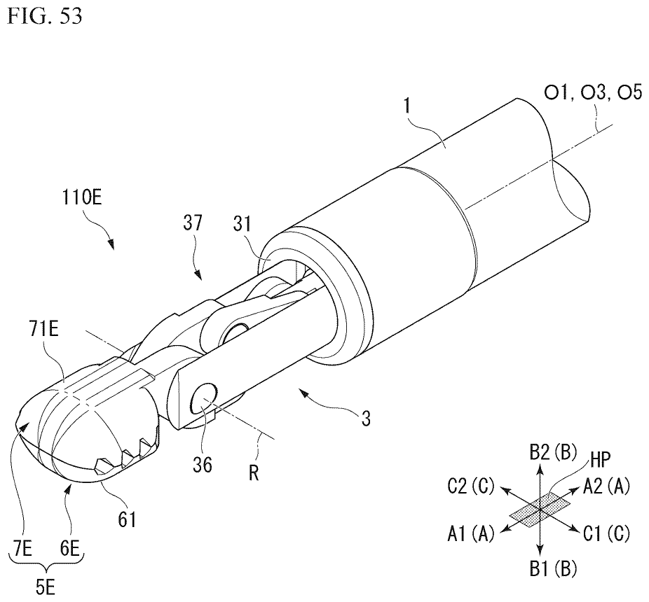

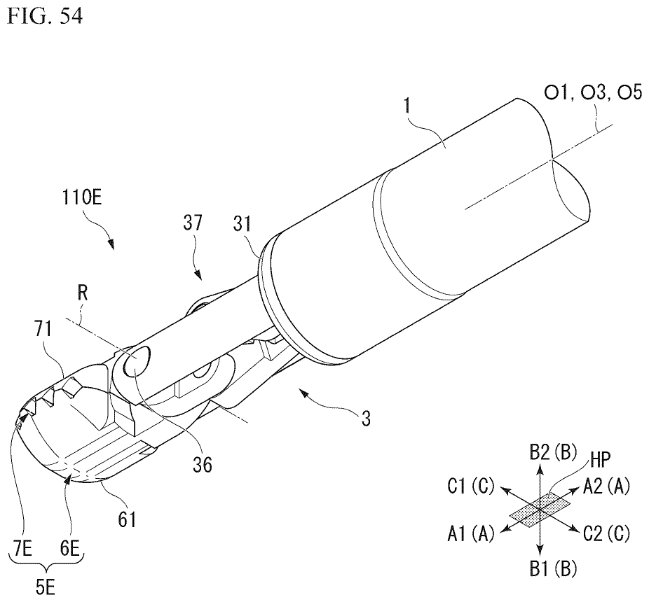

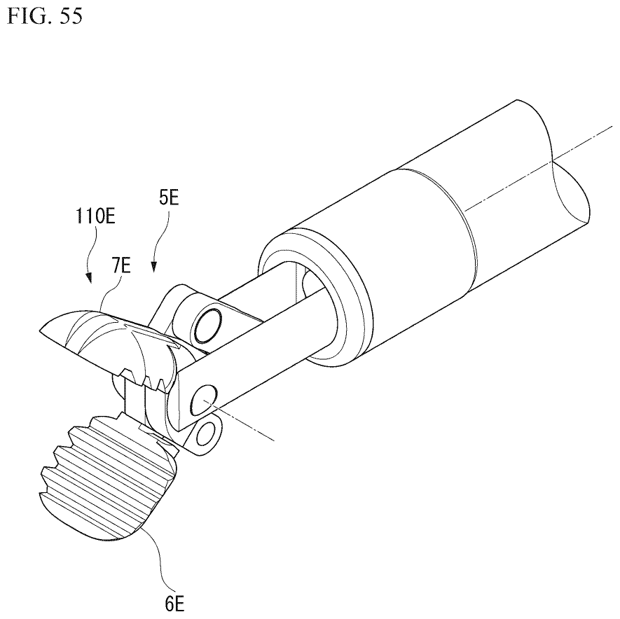

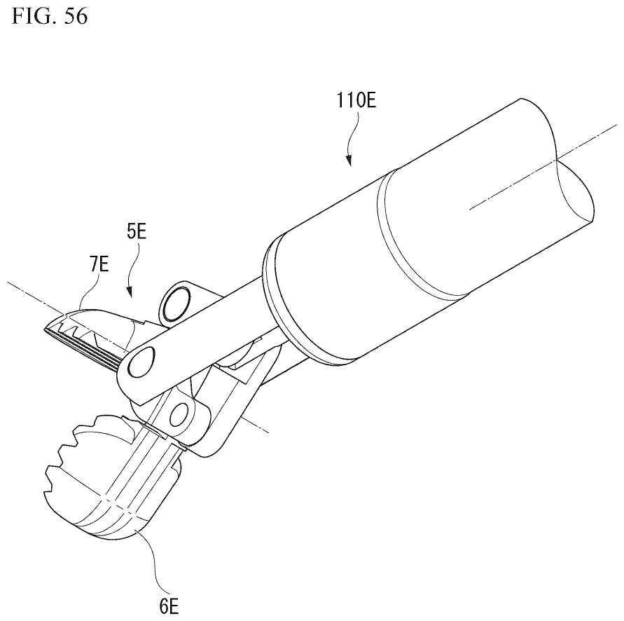

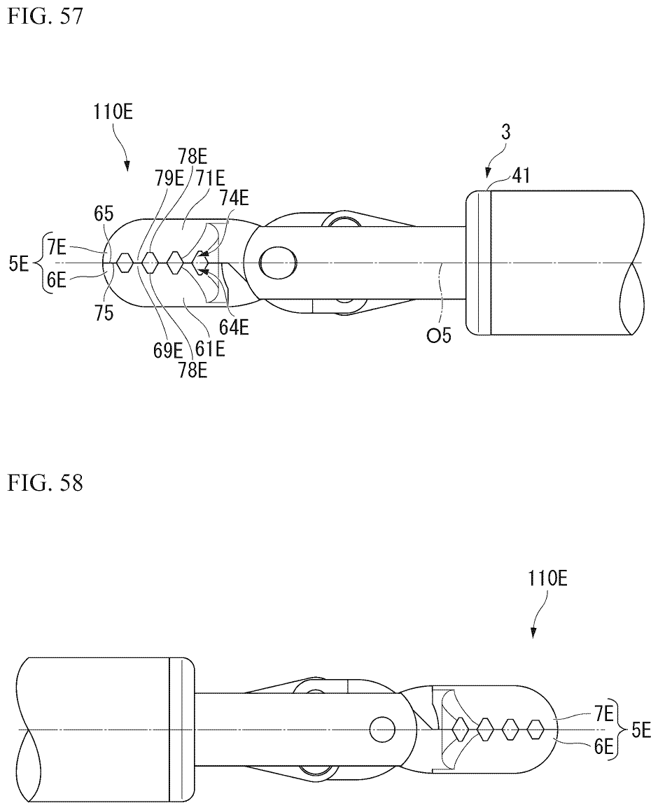

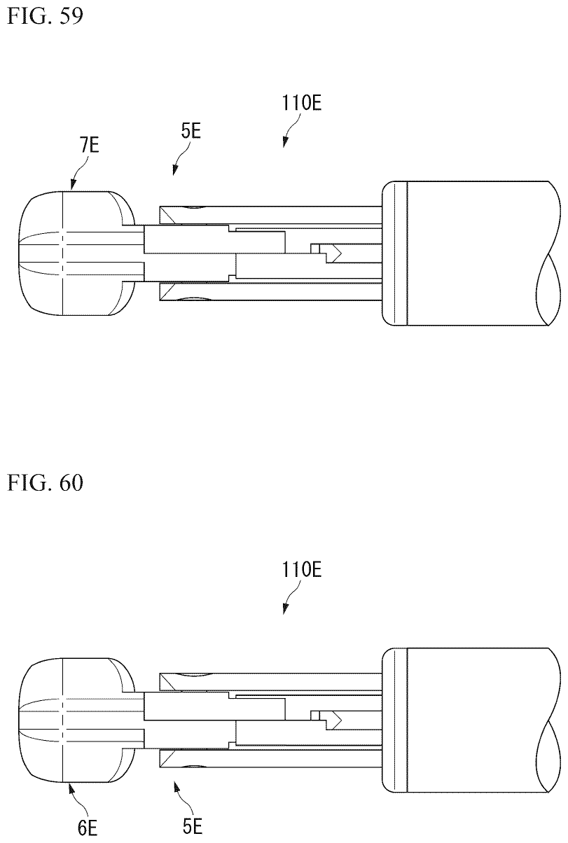

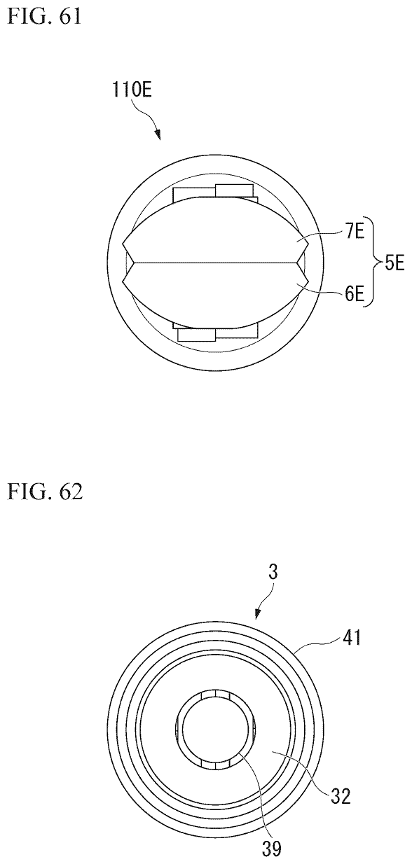

A treatment part 110E according to a fifth embodiment of the present invention will be described with reference to FIGS. 53 to 62. In the following description, components that are common to those already described will be denoted by the same reference signs and duplicate description will be omitted.

### [Endoscopic Treatment Tool 100E]

An endoscopic treatment tool 100E (also simply referred to as a treatment tool 100E) of the fifth embodiment illustrated in FIG. 1, together with an endoscope 200, is used as an endoscopic treatment system similarly to the endoscopic treatment tool 100 of the first embodiment. The treatment tool 100E includes a sheath 1, a manipulation wire 2, a support member 3, forceps 5D, and a manipulation unit 8.

### [Treatment Part 110E]

The treatment part 110E positioned at a distal end of the treatment tool 100E of the fifth embodiment includes the forceps 5E.

The forceps 5E of the present embodiment include a first forceps piece 6E and a second forceps piece 7E. When the forceps 5E are placed in a closed state, biological tissues can be gripped between a first gripping part 61E of the first forceps piece 6E and a second gripping part 71E of the second forceps piece 7E.

In the present embodiment, when the forceps 5E are in a closed state, all of tooth parts (first tooth parts) 69E of the first gripping part 61E and all of tooth parts (second tooth parts) 79E of the second gripping part 71E come into contact with each other on a gripping plane GP that passes through a central axis O5 of the forceps 5E.

Also in the present embodiment, bottom surfaces of the plurality of recessed parts 68E and 78E respectively provided in the gripping parts 61E and 71E become lower (farther away from the central axis O5) toward the proximal end side. That is, the recessed parts 68E and 78E positioned further on the proximal end side have a shape with a deeper groove, and the recessed parts 68E and 78E positioned on the most proximal end side are the deepest and are also farthest from the central axis O5.

According to the forceps 5E of the present embodiment, when the forceps 5E are in a closed state, since the recessed parts 68E and 78E that become deeper toward the proximal end side face each other, a space for accommodating a target tissue on a proximal end side (proximal side) of the forceps 5E is larger than that in the second embodiment, making it possible to grip the target tissue better without letting it escape. Also, the structure in which all the tooth parts 69E and 79E come into contact with each other makes it easier to bite into biological tissues and allows reliable gripping.

### Sixth Embodiment

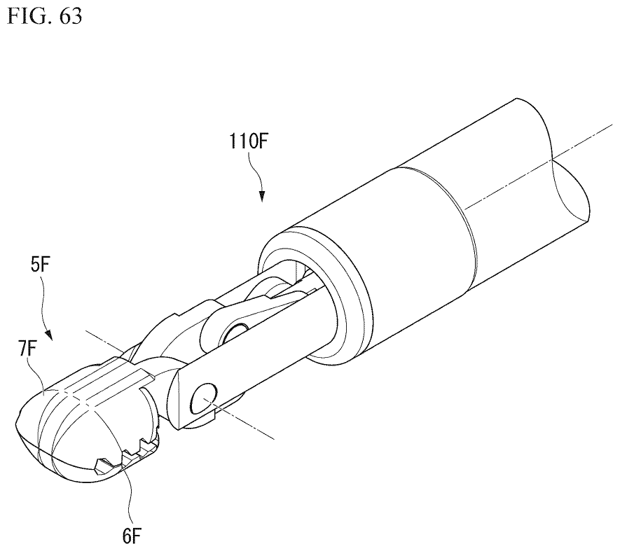

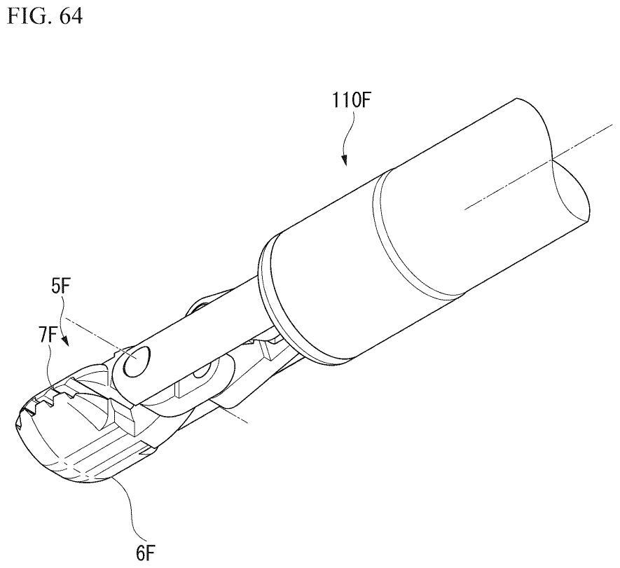

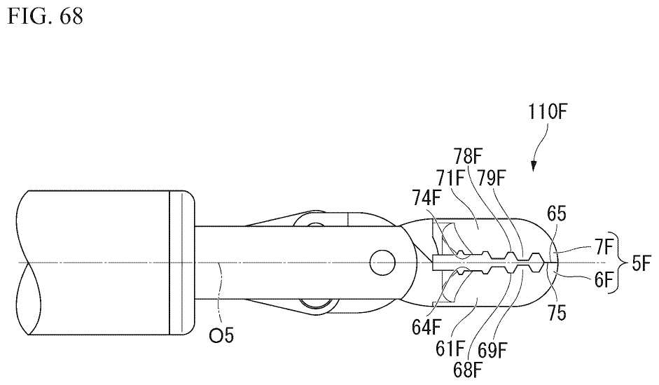

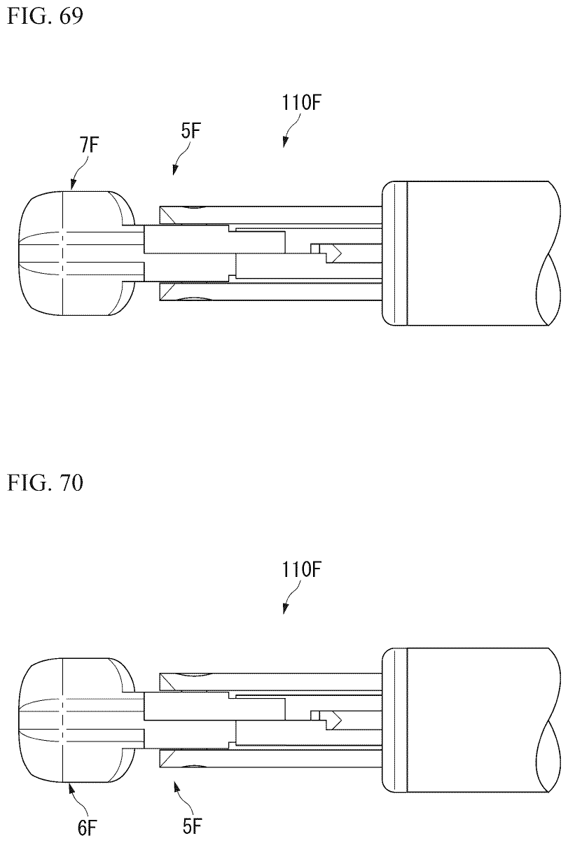

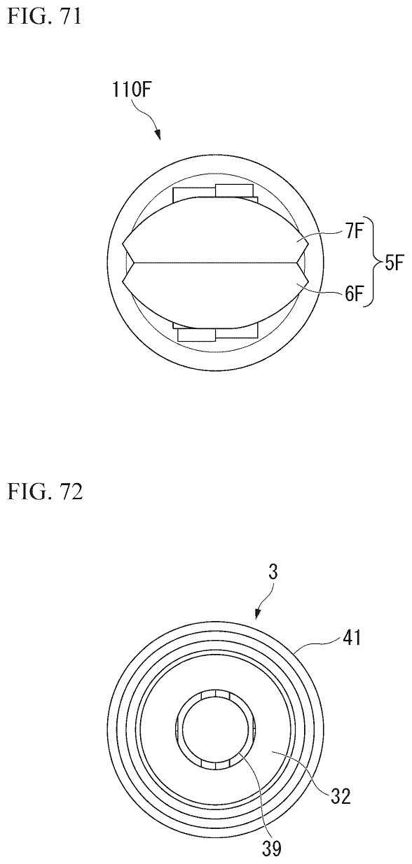

A treatment part 110F according to a sixth embodiment of the present invention will be described with reference to FIGS. 63 to 72. In the following description, components that are common to those already described will be denoted by the same reference signs and duplicate description will be omitted.

### [Endoscopic Treatment Tool 100F]

An endoscopic treatment tool 100F (also simply referred to as a treatment tool 100F) of the sixth embodiment illustrated in FIG. 1, together with an endoscope 200, is used as an endoscopic treatment system similarly to the endoscopic treatment tool 100 of the first embodiment. The treatment tool 100F includes a sheath 1, a manipulation wire 2, a support member 3, forceps 5F, and a manipulation unit 8.

### [Treatment Part 110F]

The treatment part 110F positioned at a distal end of the treatment tool 100F of the sixth embodiment includes the forceps 5F.

The forceps 5F of the present embodiment include a first forceps piece 6F and a second forceps piece 7F. When the forceps 5F are placed in a closed state, biological tissues can be gripped between a first gripping part 61F of the first forceps piece 6F and a second gripping part 71F of the second forceps piece 7F.

In the first gripping part 61F, a first gripping surface 64F is a surface with an unevenness in an opening/closing direction B, and has a plurality of recessed parts 68F and a plurality of tooth parts (first tooth parts) 69F on a proximal end side of a first contact protruding part 65 that comes into contact with a second gripping surface 74F. The recessed parts 68F and tooth parts 69F are provided alternately in a longitudinal direction A of the first gripping part 61F.

The second gripping part 71F has the second gripping surface 74F that can face the first forceps piece 6F. The second gripping surface 74F is a surface with an unevenness in the opening/closing direction B, and has a plurality of recessed parts 78F and a plurality of tooth parts (second tooth parts) 79F on a proximal end side of a second contact protruding part 75. The recessed parts 78F and the tooth parts 79F are provided alternately in the longitudinal direction A of the second gripping part 71F.

In the present embodiment, height positions of the plurality of tooth parts 69F and 79F aligned in the longitudinal direction A are different from each other, and as the tooth parts 69F and 79F are positioned further on the proximal end side, heights thereof decrease and they are positioned farther away from a central axis O5. A virtual line K6 connecting midpoints of top edges of the plurality of tooth parts 69F (79F) aligned in the longitudinal direction A is inclined at a predetermined angle with respect to the central axis O5 and forms a straight line that is lower on the proximal end side than on the distal end side.

Also, in the present embodiment, depths of the plurality of recessed parts 68F and 78F aligned in the longitudinal direction are also different, and bottom surfaces of the recessed parts 68F and 78F positioned further on the proximal end side are lower in height and are farther away from the central axis O5. A virtual line R6 connecting midpoints of the bottom surfaces of the plurality of recessed parts 68F (78F) aligned in the longitudinal direction A is inclined at a predetermined angle with respect to the central axis O5, and forms an inclined surface that is lower on the proximal end side than on the distal end side.

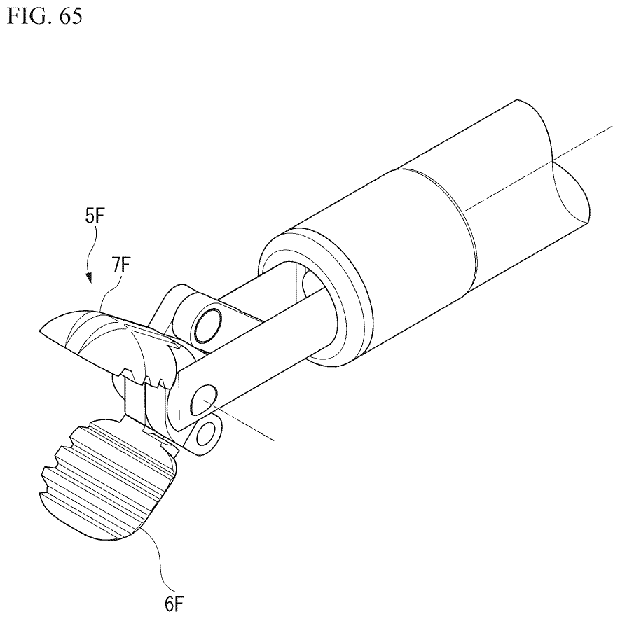

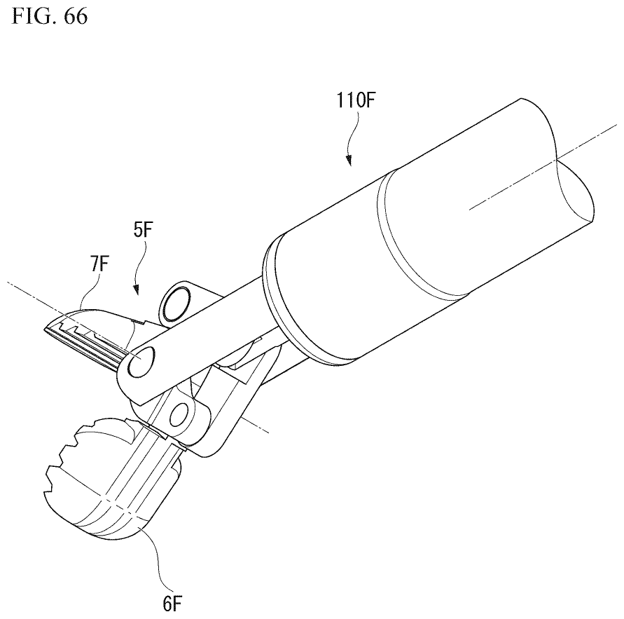

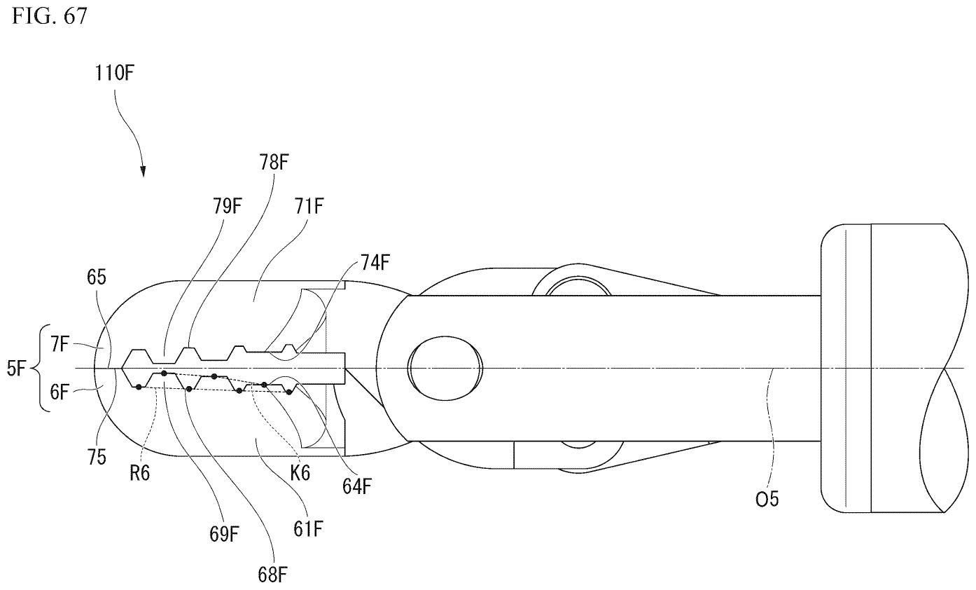

As illustrated in FIG. 67, an angle formed by the virtual line R6 connecting the midpoints of the bottom surfaces of the plurality of recessed parts 68F aligned in the longitudinal direction A and the central axis O5 is smaller than an angle formed by the virtual line K6 connecting the midpoints of the top edges of the plurality of tooth parts 69F aligned in the longitudinal direction A and the central axis O5. That is, a gradient of the bottom surfaces of the recessed parts 68F and 78F is smaller than a gradient of the tooth surfaces of the tooth parts 69F and 79F. Therefore, as it comes closer to the proximal end side, depths of the recessed parts 68F and 78F become smaller, and a distance between the facing forceps pieces 6F and 7F increases.

According to the forceps 5F of the present embodiment, when the forceps 5F are in a closed state, since the uneven shape becomes smaller toward the proximal end side, a space for accommodating a target tissue on the proximal end side (proximal side) of the forceps 5F is larger than that in the second embodiment, making it possible to grip the target tissue better without letting it escape.

### Seventh Embodiment

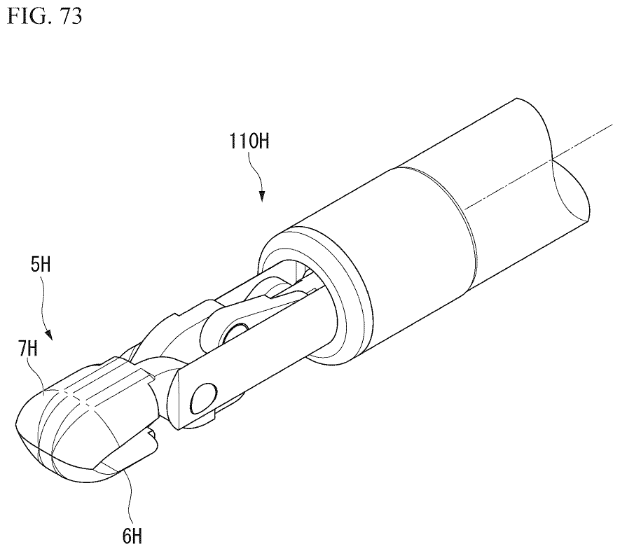

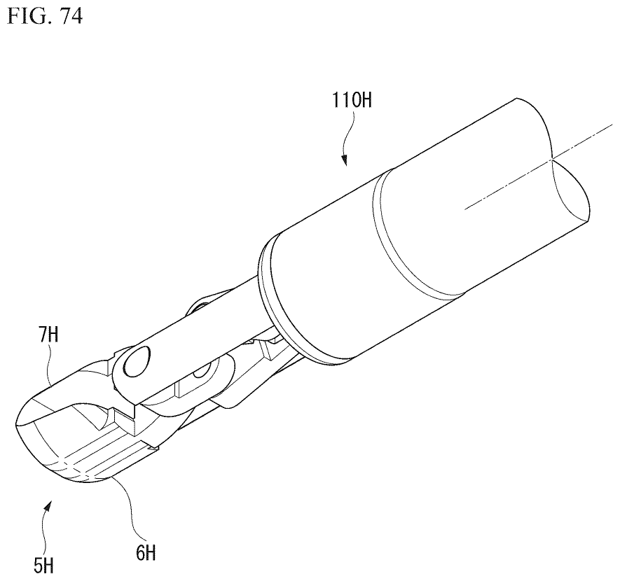

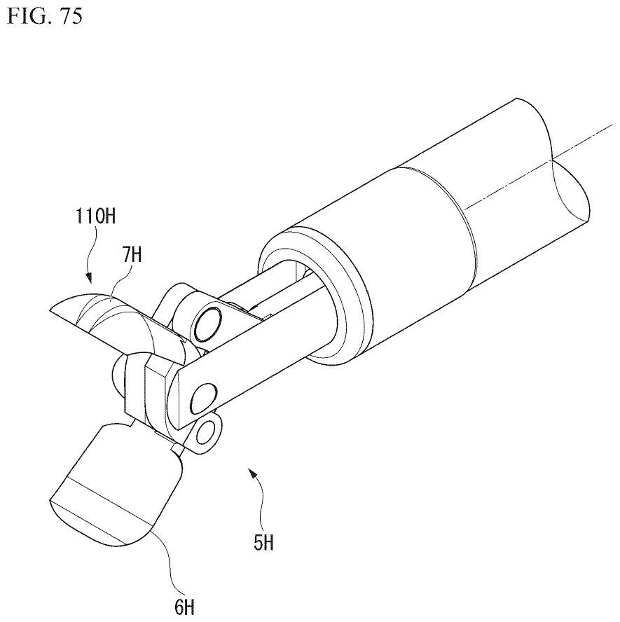

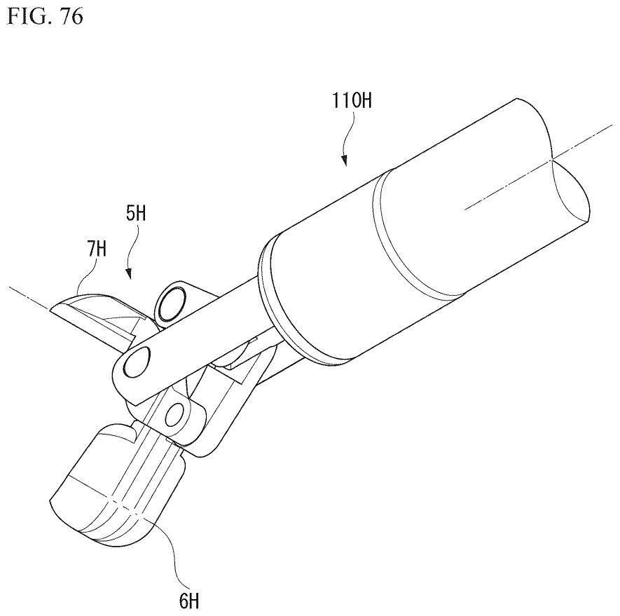

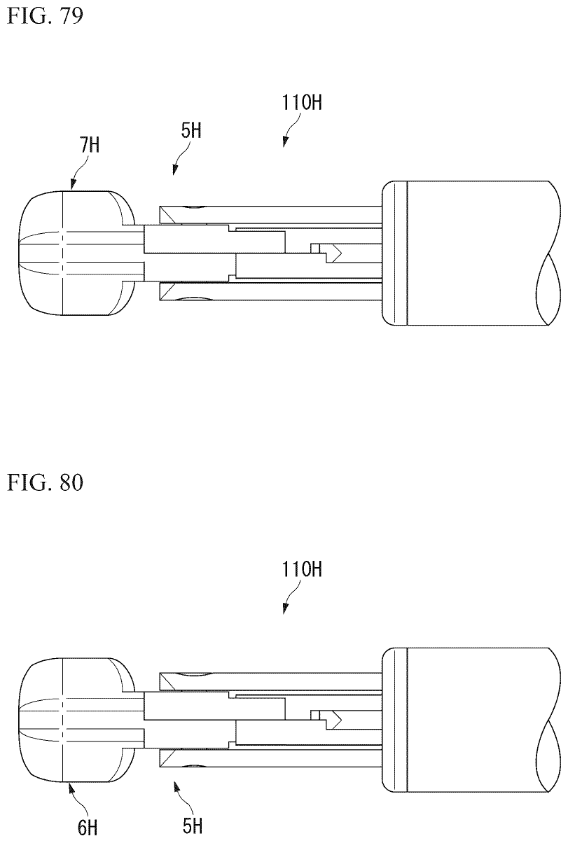

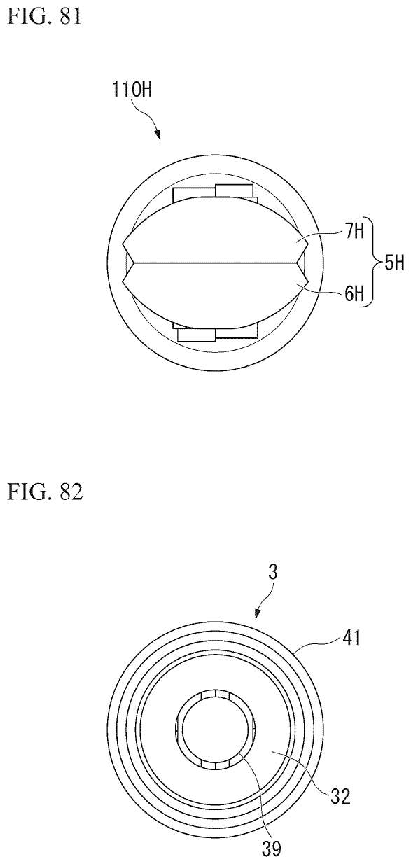

A treatment part 110H according to a seventh embodiment of the present invention will be described with reference to FIGS. 73 to 82. In the following description, components that are common to those already described will be denoted by the same reference signs and duplicate description will be omitted.

### [Endoscopic Treatment Tool 100H]

An endoscopic treatment tool 100H (also simply referred to as a treatment tool 100H) of the seventh embodiment illustrated in FIG. 1, together with an endoscope 200, is used as an endoscopic treatment system similarly to the endoscopic treatment tool 100 of the first embodiment. The treatment tool 100H includes a sheath 1, a manipulation wire 2, a support member 3, forceps 5H, and a manipulation unit 8.

[Treatment part 110H]

The treatment part 110H positioned at a distal end of the treatment tool 100H of the eighth embodiment includes the forceps 5H.

The forceps 5H of the present embodiment include a first forceps piece 6H and a second forceps piece 7H. When the forceps 5H are placed in a closed state, biological tissues can be gripped between a first gripping part 61H of the first forceps piece 6H and a second gripping part 71H of the second forceps piece 7H.

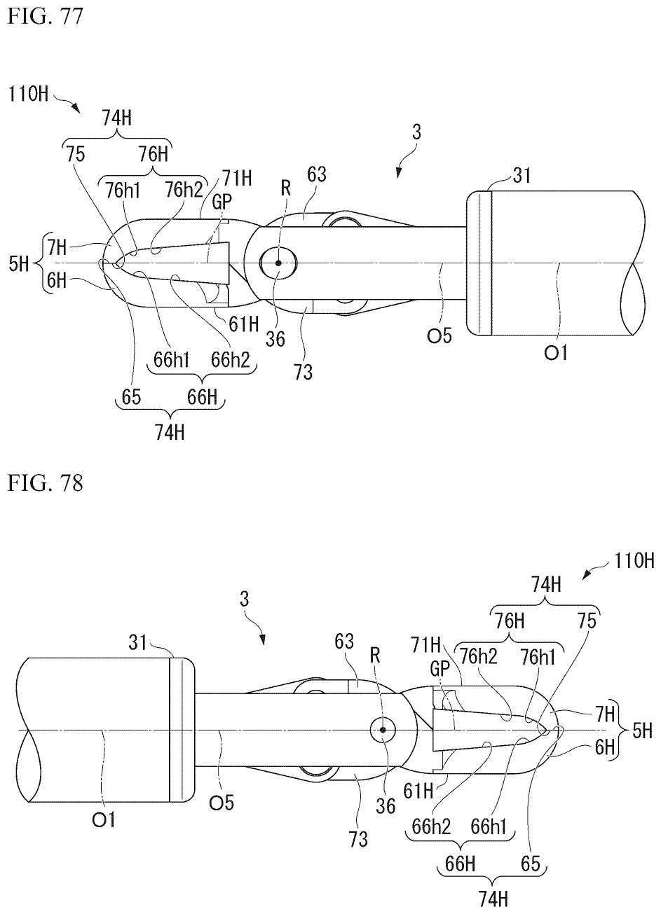

As illustrated in FIGS. 77 and 78, the first gripping part 61H has a first gripping surface 64H that can face the second forceps piece 7H. Also, the second gripping part 71H has a second gripping surface 74H that can face the first forceps piece 6H. The first gripping surface 64H and the second gripping surface 74H are surfaces facing each other when the forceps 5H are in a closed state, and have contact protruding parts 65 and 75 that come into contact with each other on a gripping plane GP passing through a central axis O5 of the forceps 5H when the forceps 5H are in a closed state, accommodating recessed parts 66H and 76H that do not come into contact, and notch parts 67 and 77 provided on a distal end side of the accommodating recessed parts 66H and 76H.

The accommodating recessed parts 66H and 76H are recessed parts recessed in a direction away from the central axis O5 when the forceps 5H are in a closed state. The accommodating recessed parts 66H and 76H gradually increase in groove depth from a proximal end side of the contact protruding parts 65 and 75 toward a proximal end side of the forceps 5H. The accommodating recessed parts 66H and 77H have curved surfaces 66h1 and 76h1 respectively positioned on a distal end side (the contact protruding parts 65 and 75 side) and inclined surfaces 66h2 and 76h2 respectively positioned on a proximal end side of the curved surfaces 66h1 and 76h1.

Of a length of the accommodating recessed parts 66H and 76H in a longitudinal direction A, the curved surfaces 66h1 and 76h1 occupy about one-quarter of the length from the distal end side. Since about three-quarters of the remaining length is occupied by the inclined surfaces 66h2 and 76h2, a middle portion of the forceps 5H forms a tapered shape that widens toward the proximal end side due to the inclined surfaces 66h2 and 76h2.

The inclined surfaces 66h2 and 76h2 are inclined at a predetermined angle with respect to the central axis O5. In the present embodiment, an inclination angle θ6 of the inclined surface 66h2 with respect to the central axis O5 and an inclination angle θ7 of the inclined surface 67h2 with respect to the central axis O5 are equal to each other, but they may be different.

According to the forceps 5H of the present embodiment, since groove depths of the accommodating recessed parts 66H and 76H increase and a distance between the facing gripping surfaces 64H and 74H increases as it comes closer to the proximal end side, a space for accommodating a target tissue on the proximal end side (proximal side) of the forceps 5H is larger than that in the first embodiment described above, and when the forceps 5H is placed in a closed state and the target tissue is gripped between the contact protruding parts 65 and 75 at the distal end, the target tissue becomes more difficult to escape.

Also, a part of the target tissue gripped by the contact protruding parts 65 and 75 slightly enters the notch parts 67 and 77 provided at distal ends of the gripping parts 61H and 71H, causing a state in which the target tissue is caught by the gripping parts 61H and 71H, making it possible to grip the target tissue better without letting it escape.

### Eighth Embodiment

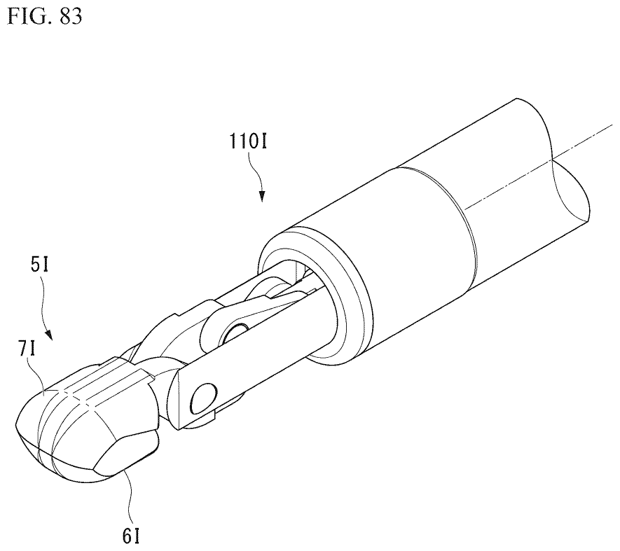

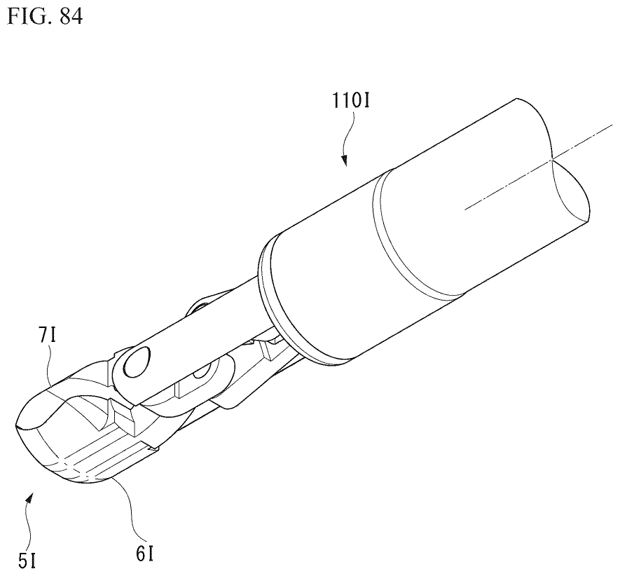

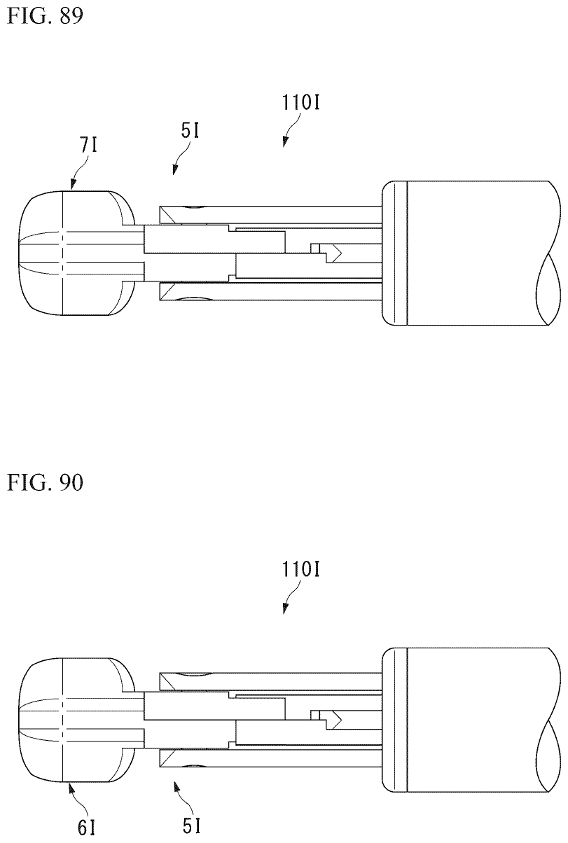

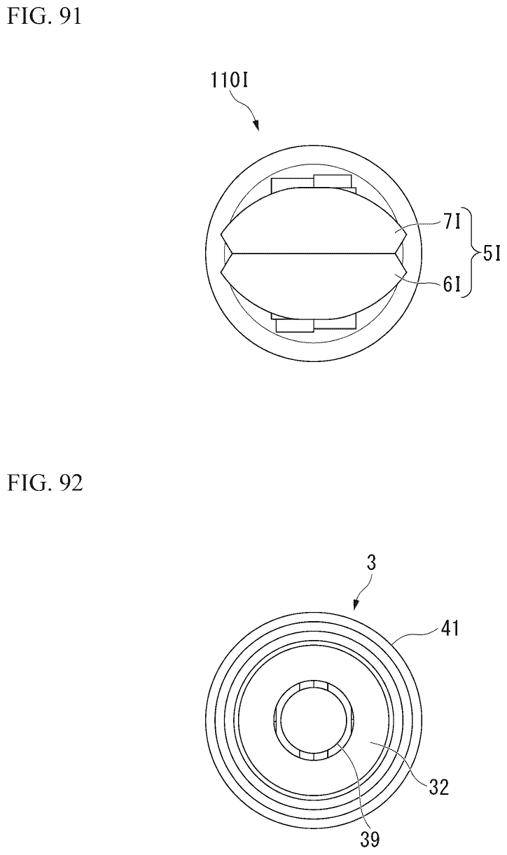

A treatment part 110I according to an eighth embodiment of the present invention will be described with reference to FIGS. 83 to 92. In the following description, components that are common to those already described will be denoted by the same reference signs and duplicate description will be omitted.

### [Endoscopic Treatment Tool 100I]

An endoscopic treatment tool 100I (also simply referred to as a treatment tool 100I) of the eighth embodiment illustrated in FIG. 1, together with an endoscope 200, is used as an endoscopic treatment system similarly to the endoscopic treatment tool 100 of the first embodiment. The treatment tool 100I includes a sheath 1, a manipulation wire 2, a support member 3, forceps 5I, and a manipulation unit 8.

[Treatment part 110I]

The treatment part 110I positioned at a distal end of the treatment tool 100I of the eighth embodiment includes the forceps 5I.

The forceps 5I of the present embodiment include a first forceps piece 6I and a second forceps piece 7I. When the forceps 5I are placed in a closed state, biological tissues can be gripped between a first gripping part 61I of the first forceps piece 6I and a second gripping part 71I of the second forceps piece 7I.

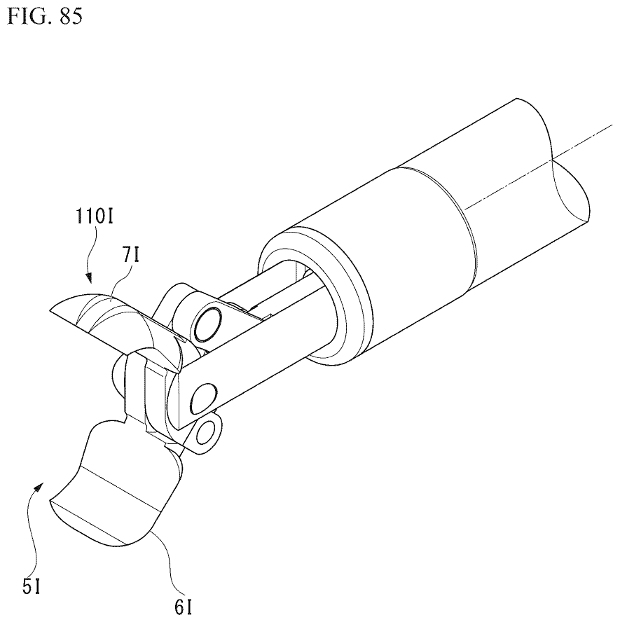

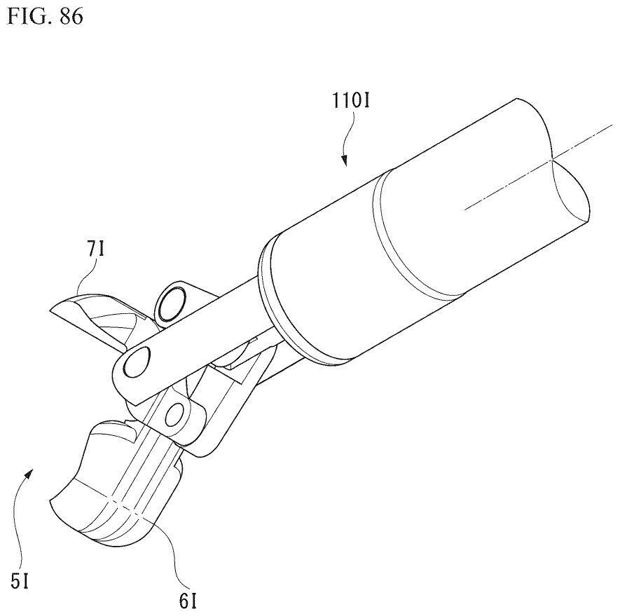

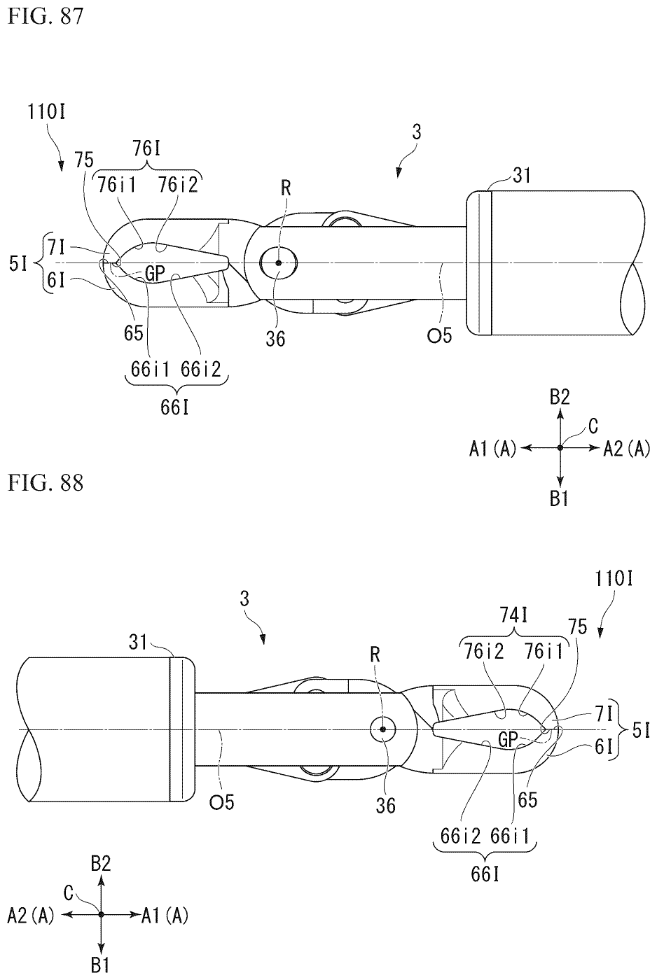

As illustrated in FIGS. 87 and 88, the first gripping part 61I has a first gripping surface 64I that can face the second forceps piece 7I. The second gripping part 71I has a second gripping surface 74I that can face the first forceps piece 6I. The first gripping surface 64I and the second gripping surface 74I are surfaces facing each other when the forceps 5I are in a closed state, and have contact protruding parts 65 and 75 that come into contact with each other on a gripping plane GP passing through a central axis O5 of the forceps 5I when the forceps 5I are in a closed state, and accommodating recessed parts 66I and 76I that do not come into contact.

The accommodating recessed parts 66I and 76I are recessed parts recessed from the gripping plane GP to a lower side B1 or an upper side B2 when the forceps 5I are in a closed state, and are provided on a proximal end side with respect to the contact protruding parts 65 and 75. A groove depth of the accommodating recessed parts 66I and 76I varies in a longitudinal direction A. In the present embodiment, the groove depth of the accommodating recessed parts 66I and 76I is largest at a position on a front end side past halfway along a length of the accommodating recessed parts 66I and 76I in the longitudinal direction A, and becomes smaller toward a rear end side.

The accommodating recessed part 66I has a curved surface 6611 positioned on a front end side and an inclined surface 6612 positioned on a rear end side, and a boundary between them is smoothly connected without any step. Also, the accommodating recessed part 76I has a curved surface 76i1 positioned on the front end side and an inclined surface 7612 positioned on the rear end side, and a boundary between them is smoothly connected without any step.

The curved surfaces 6611 and 7611 are circular arcs centered on points (not illustrated) positioned on the sides of the gripping parts 61I and 71I facing each other, and are curved more largely toward the lower side B1 or the upper side B2 compared to those in the first embodiment.

The inclined surfaces 6612 and 7612 are connected to the proximal end sides of the curved surfaces 6611 and 7611, and are formed in a substantially planar shape on the lower side B1 or the upper side B2 of the gripping plane GP. The inclined surfaces 6612 and 7612 are inclined at a predetermined angle with respect to the central axis O5 and come closer to each other toward the proximal end side.

According to the forceps 5I of the present embodiment, since the groove depth of the accommodating recessed parts 66I and 76I is larger on the distal end (the contact protruding parts 65 and 75) side than on the proximal end side, when biological tissues of the target tissue are gripped by the first gripping surface 64I and the second gripping surface 74I of the forceps 5I, a vicinity of the portion gripped by the contact protruding parts 65 and 75 can be accommodated in the accommodating recessed parts 66I and 76I. Thereby, it is more difficult for the biological tissues to be push out to a distal end side (distal side) A1, making it easier to grip the biological tissues to be treated more stably.

### Ninth Embodiment

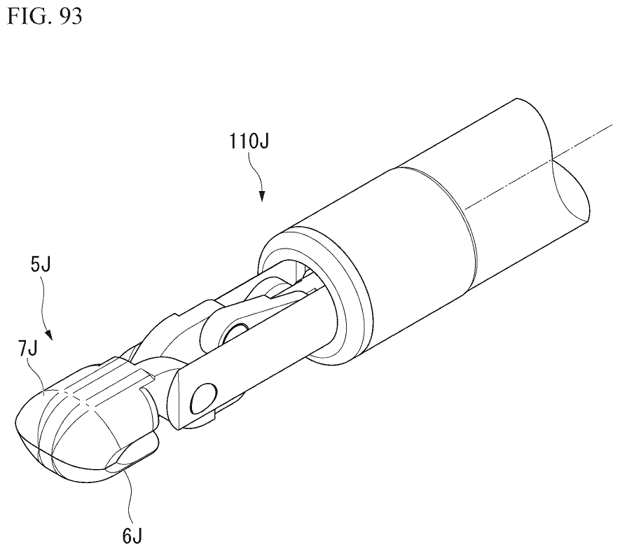

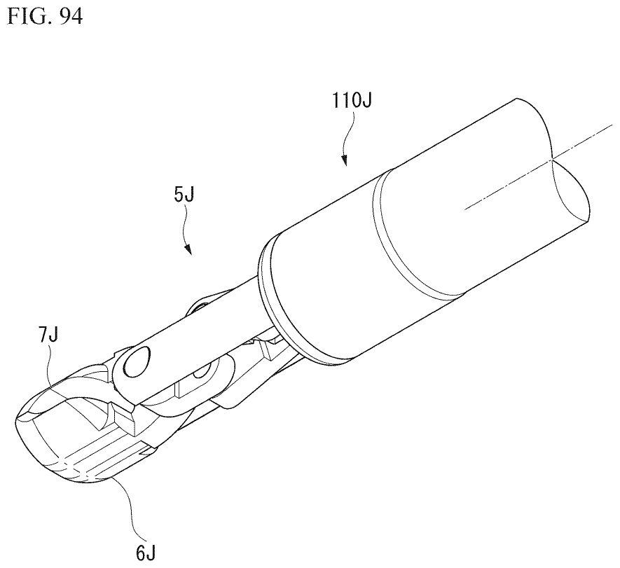

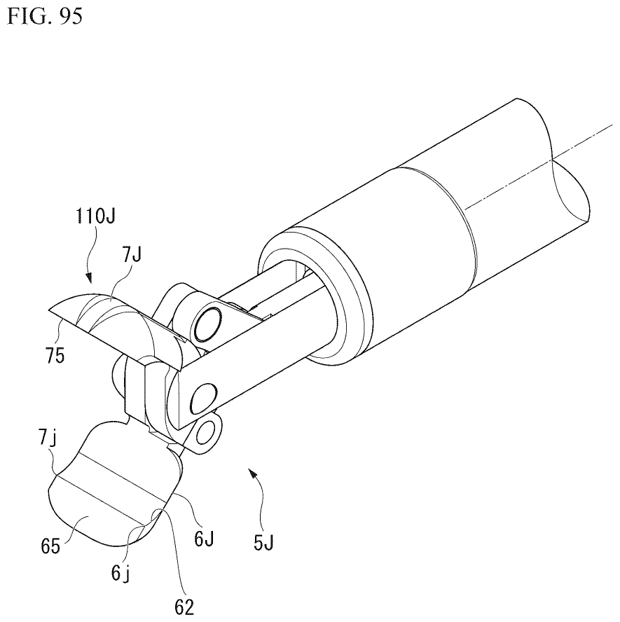

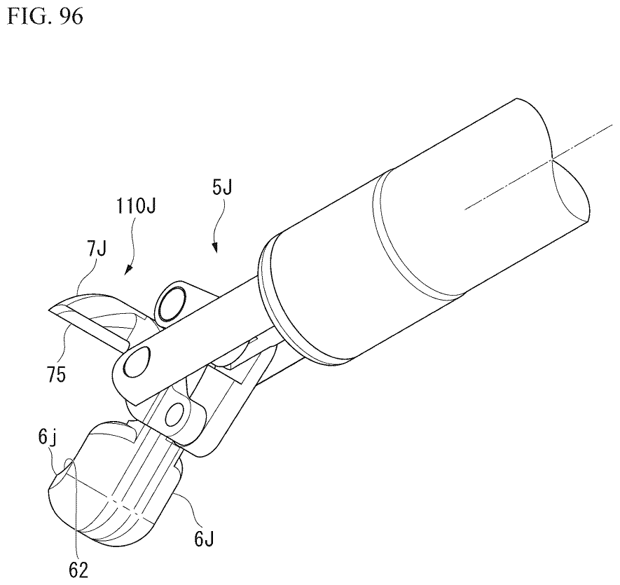

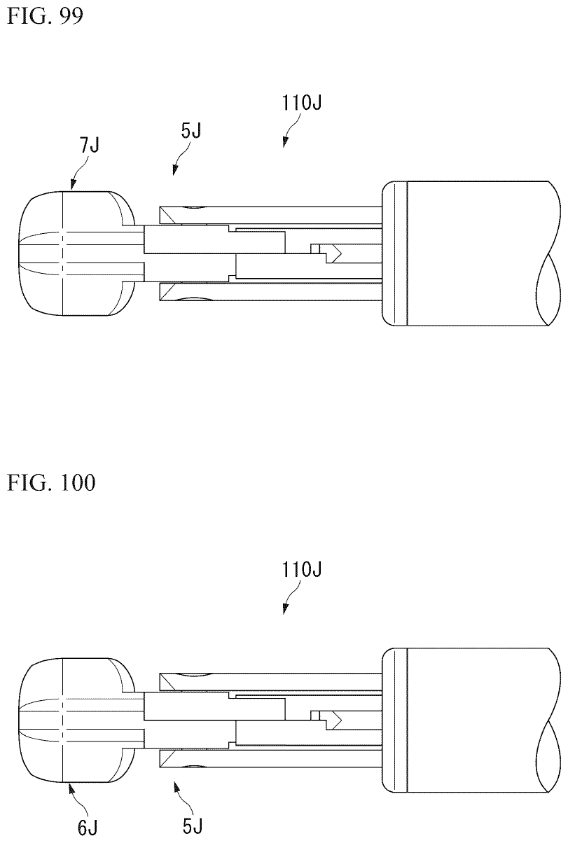

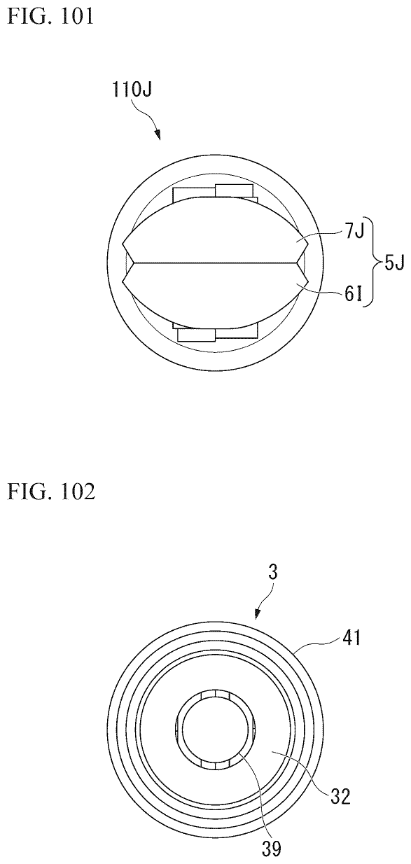

A treatment part 110J according to a ninth embodiment of the present invention will be described with reference to FIGS. 93 to 102. In the following description, components that are common to those already described will be denoted by the same reference signs and duplicate description will be omitted.

### [Endoscopic Treatment Tool 100J]

An endoscopic treatment tool 100J (also simply referred to as a treatment tool 100J) of the ninth embodiment illustrated in FIG. 93, together with an endoscope 200, is used as an endoscopic treatment system similarly to the endoscopic treatment tool 100 of the first embodiment. The treatment tool 100J includes a sheath 1, a manipulation wire 2, a support member 3, forceps 5J, and a manipulation unit 8.

### [Treatment Part 110J]

The treatment part 110J positioned at a distal end of the treatment tool 100J of the ninth embodiment includes the forceps 5J.

The forceps 5J of the present embodiment include a first forceps piece 6J and a second forceps piece 7J. When the forceps 5J are placed in a closed state, biological tissues can be gripped between a first gripping part 61J of the first forceps piece 6J and a second gripping part 71J of the second forceps piece 7J.

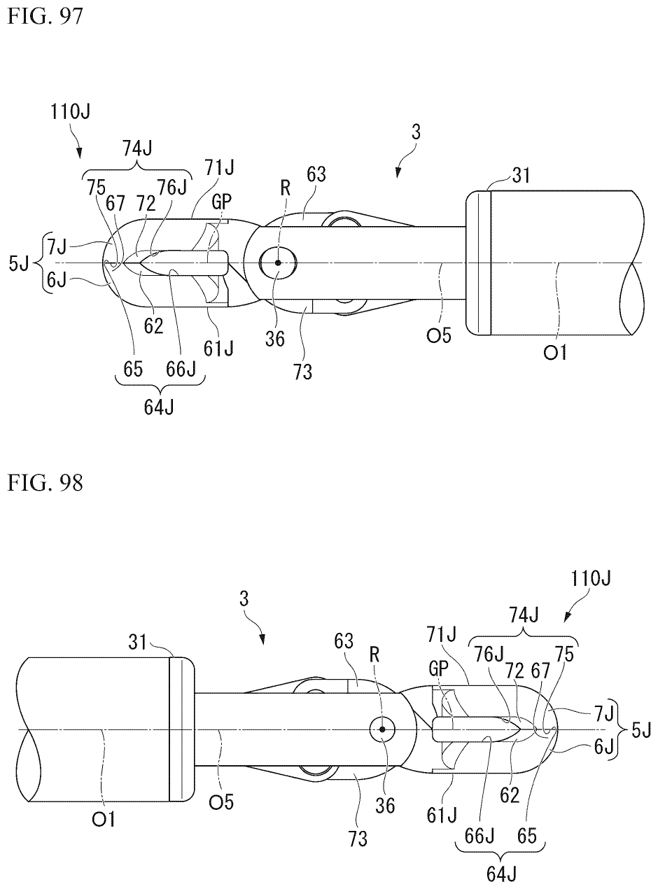

As illustrated in FIGS. 97 and 98, the first gripping part 61J has a first gripping surface 64J that can face the second forceps piece 7J. The second gripping part 71J has a second gripping surface 74J that can face the first forceps piece 6J. The first gripping surface 64J and the second gripping surface 74J are surfaces facing each other when the forceps 5J are in a closed state, and have contact protruding parts 65 and 75 that come into contact with each other on a gripping plane GP passing through a central axis O5 of the forceps 5J when the forceps 5J are in a closed state, and accommodating recessed parts 66J and 76J that do not come into contact.

In the present embodiment, an area of the gripping plane GP, where the forceps 5J face each other in a closed state, is larger than that of the embodiments described above. Specifically, the contact protruding parts 65 and 75 in the forceps 5J of the present embodiment have a length in a longitudinal direction A, and a depth that is about twice that of the embodiments described above. With this configuration, an engagement area of the forceps 5J increases, making it possible to widen a cauterization range.

The accommodating recessed parts 66J and 76J are recessed parts recessed from the gripping plane GP to a lower side B1 or an upper side B2 when the forceps 5J are in a closed state, and are provided on a proximal end side with respect to the contact protruding parts 65 and 75. A groove depth of the accommodating recessed parts 66J and 76J varies in the longitudinal direction A. In the present embodiment, the groove depth of the accommodating recessed parts 66J and 76J becomes larger from a proximal end side of the contact protruding parts 65 and 75 toward a position about halfway along a length of the accommodating recessed parts 66J and 76J in the longitudinal direction A, and is constant from a deepest position thereof to a rear end. That is, the proximal end side of the accommodating recessed parts 66J and 76J is formed in a substantially planar shape.

Also, notched surfaces 62 and 72 that are cut inward in a width direction from an outer circumferential surface are formed on both sides in the width direction of the first gripping part 61J and the second gripping part 71J. Due to provision of the notched surfaces 62 and 72, corners (apexes) 6j and 7j formed at a boundary portion between the contact protruding parts 65 and 75 and flat surfaces of the contact protruding parts 65 and 75 are positioned within the forceps 5J. Thereby, during an opening and closing motion of the forceps 5J, the corners 6j and 7j do not get caught on biological tissues around the target tissue, and the opening and closing motion of the forceps 5J can be smoothly performed.

### Tenth Embodiment

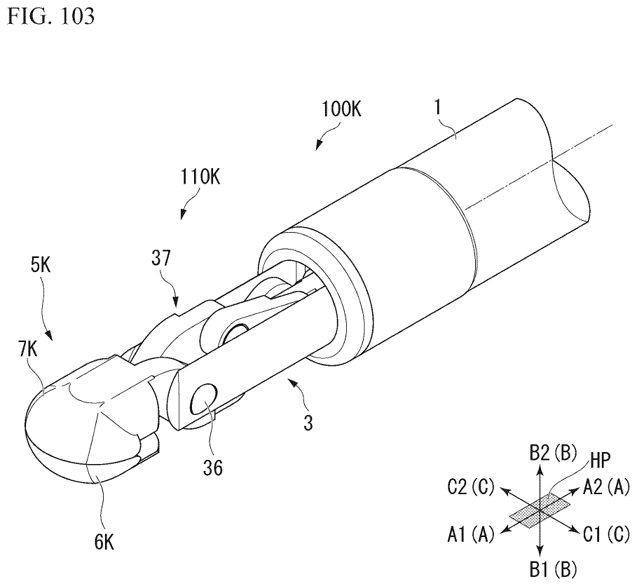

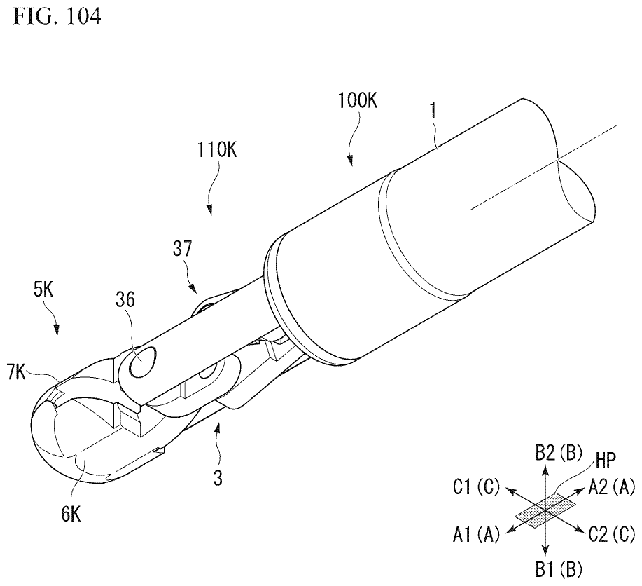

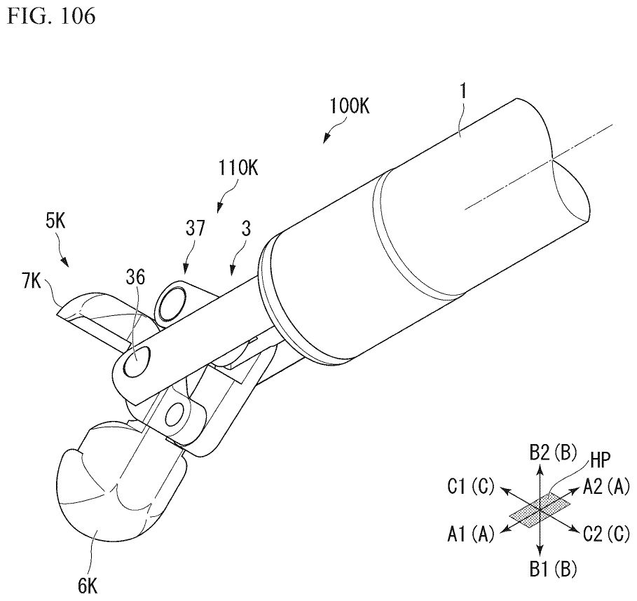

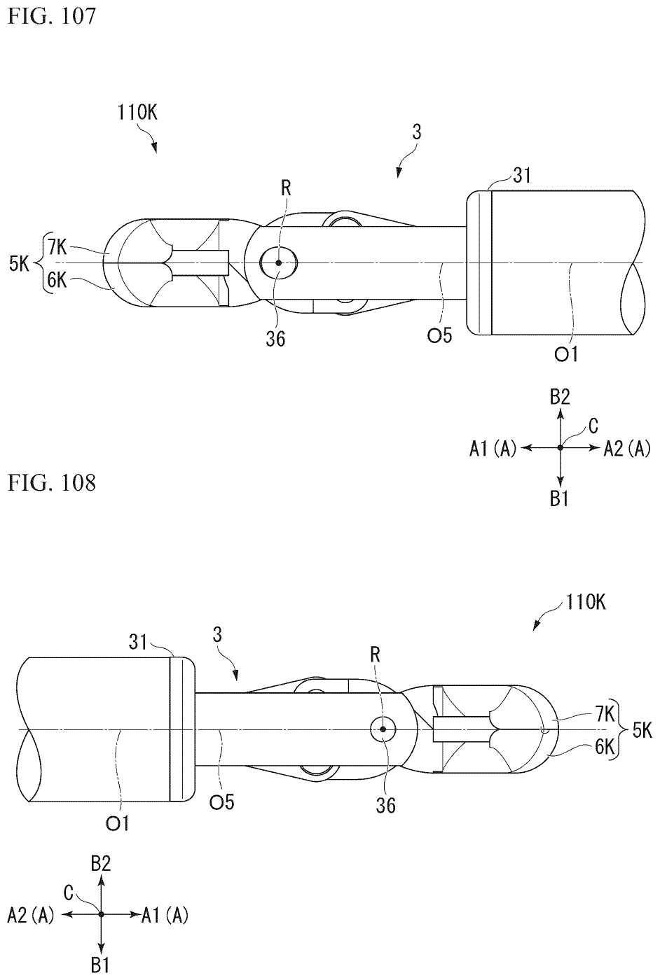

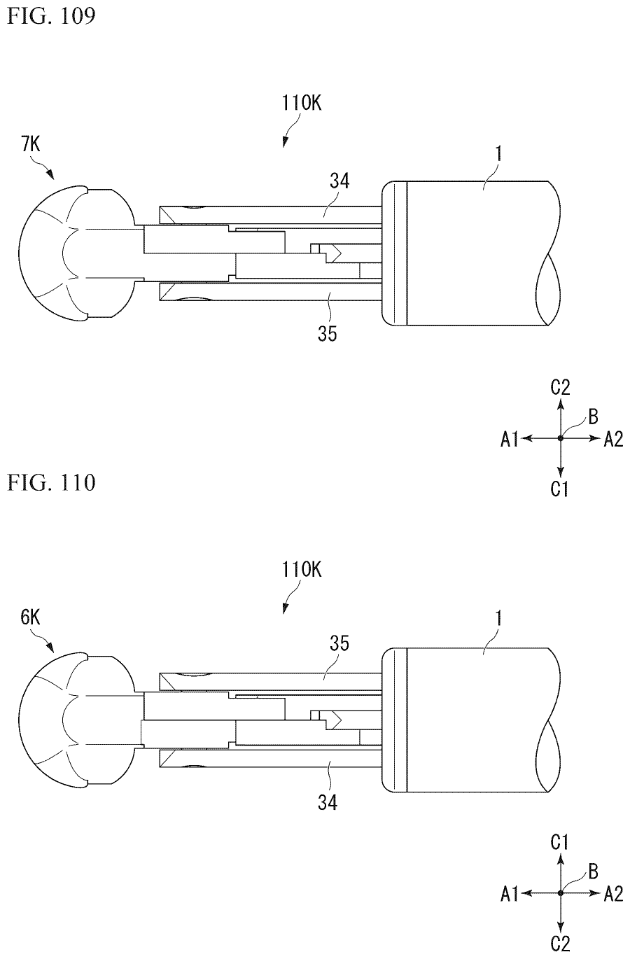

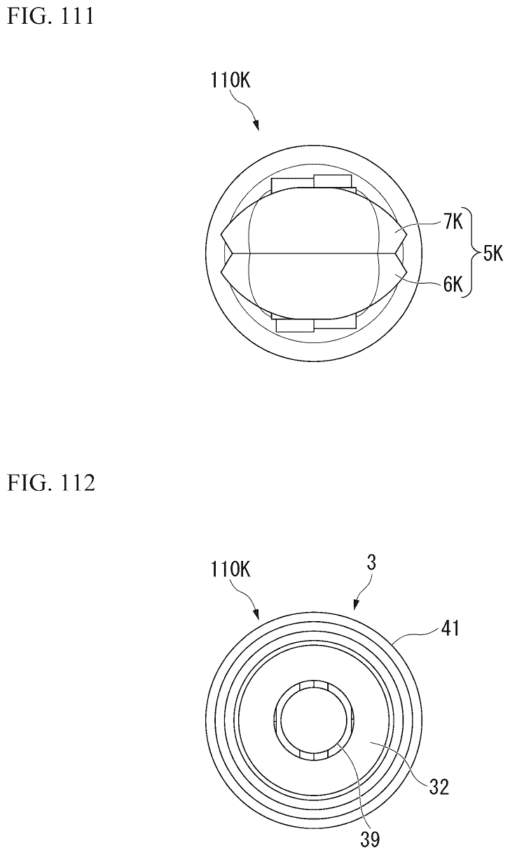

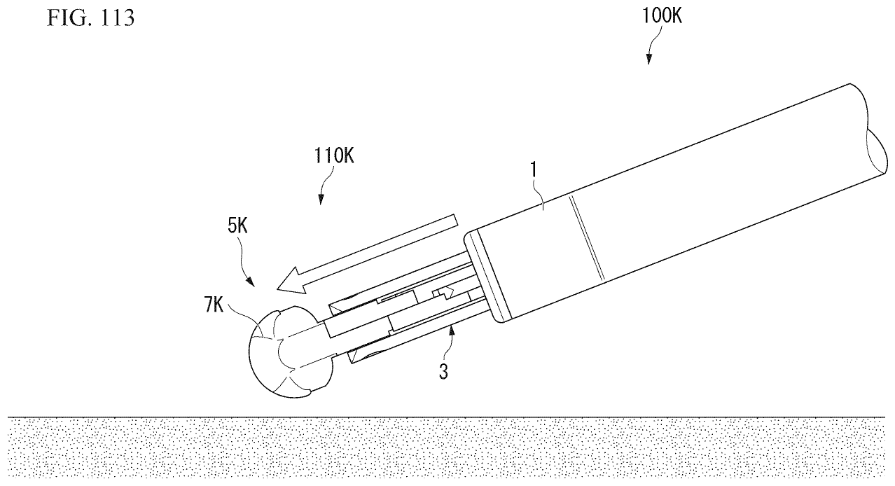

A treatment part 110K according to a tenth embodiment of the present invention will be described with reference to FIGS. 103 to 113. In the following description, components that are common to those already described will be denoted by the same reference signs and duplicate description will be omitted.

### [Endoscopic Treatment Tool 100K]

An endoscopic treatment tool 100K (also simply referred to as a treatment tool 100K) of the tenth embodiment illustrated in FIG. 103, together with an endoscope 200, is used as an endoscopic treatment system similarly to the endoscopic treatment tool 100 of the first embodiment. The treatment tool 100K includes a sheath 1, a manipulation wire 2, a support member 3, forceps 5K, and a manipulation unit 8.

### [Treatment Part 110K]

The treatment part 110K positioned at a distal end of the treatment tool 100K of the tenth embodiment includes the forceps 5K.

The forceps 5K of the present embodiment includes a first forceps piece 6K and a second forceps piece 7K. When the forceps 5K are placed in a closed state, biological tissues can be gripped between a first gripping part 61K of the first forceps piece 6K and a second gripping part 71K of the second forceps piece 7K.

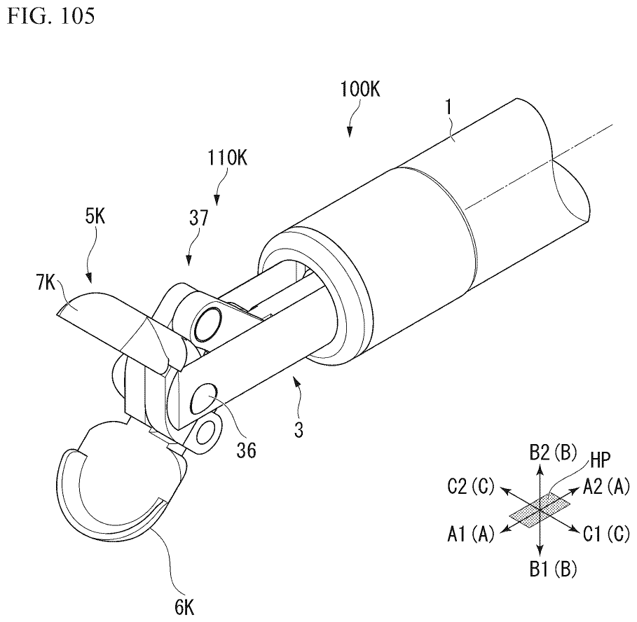

In the present embodiment, as illustrated in FIG. 105, an outer shape of the first forceps piece 6K of a distal end side A1 is semicircular, and a tooth provided on the distal end side A1 of the first forceps piece 6K is arc-shaped to follow the outer shape. Further, an outer shape of the second forceps piece 7K on the distal end side A1 is semicircular, and a tooth provided on the distal end side A1 of the second forceps piece 7K is arc-shaped to follow the outer shape. Therefore, as illustrated in FIG. 113, when a target tissue is brought close to a side surface of the forceps 5K, the target tissue can be easily gripped by the first forceps piece 6K and the second forceps piece 7K.

While preferred embodiments of the present invention have been described above, the present invention is not limited to these embodiments. Additions, omissions, substitutions, and other changes to the configurations can be made without departing from the spirit of the present invention.

Also, the present invention is not to be considered as being limited by the foregoing description and is only limited by the scope of the appended claims.

As illustrated in the front views of the embodiments described above, when the forceps are viewed from the distal end (A2) side in the longitudinal direction A, dimensions in the width direction C of the contact protruding parts 65 and 75 are equal to each other, and both are smaller than a maximum width dimension in the width direction C of the forceps.

### INDUSTRIAL APPLICABILITY

The present invention can be applied to an endoscopic treatment tool such as hemostatic forceps.

What is claimed is:

1. A treatment tool comprising:
a sheath; and
forceps having a first forceps piece provided on a distal end of the sheath and a second forceps piece configured to open and close with respect to the first forceps piece, wherein
the first forceps piece has a plurality of first tooth parts protruding toward the second forceps piece and a plurality of first recessed parts alternately disposed in a central axis direction, and
in a side view from a direction perpendicular to a central axis of the sheath, when the first forceps piece and the second forceps piece are closed, an angle formed by a straight line (R6) connecting midpoints of bottom surfaces of the plurality of first recessed parts and the central axis is smaller than an angle formed by a straight line (K6) connecting midpoints of top edges of the plurality of first tooth parts and the central axis.

2. The treatment tool according to claim 1, wherein
the second forceps piece has a plurality of second tooth parts protruding toward the first forceps piece and a plurality of second recessed parts alternately disposed in the central axis direction, and
in the side view from the direction perpendicular to the central axis of the sheath, when the first forceps piece and the second forceps piece are closed, an angle formed by a straight line connecting midpoints (R6) of bottom surfaces of the plurality of second recessed parts and the central axis is smaller than an angle formed by a straight line (K6) connecting midpoints of top edges of the plurality of first tooth parts and the central axis.

3. The treatment tool according to claim 1, wherein the length in the width direction of the first forceps piece and the second forceps piece is equal to or less than a maximum outer diameter of the sheath.

4. The treatment tool according to claim 1, wherein a length in the width direction of the forceps is 1.0 times or more and 1.6 times or less a length in the longitudinal direction of the forceps.

5. The treatment tool according to claim 1, wherein an opening width of the forceps in a state of being opened to the maximum is 1.7 times or more and 2.8 times or less a length in the longitudinal direction of the forceps.

6. The treatment tool according to claim 1, wherein a length of the forceps in a thickness direction orthogonal to the longitudinal direction when the forceps are closed is 0.2 times or more to 0.6 times or less a length in the longitudinal direction of the forceps.

7. The treatment tool according to claim 1, wherein a length in the width direction of the forceps is 1.1 times a length in the longitudinal direction of the forceps.

8. The treatment tool according to claim 1, wherein an opening width of the forceps in a state of being opened to the maximum is 2.0 times a length of a forceps piece in a longitudinal axial direction.

9. The treatment tool according to claim 1, wherein a length of the forceps in a thickness direction orthogonal to the longitudinal direction when the forceps are closed is 0.3 times a length of a forceps piece in the longitudinal direction.

10. The treatment tool according to claim 1, wherein
in the side view from the direction perpendicular to a central axis of the sheath, when the first forceps piece and the second forceps piece are closed, the top edges of the plurality of first tooth parts extend parallel with the central axis of the sheath.
